(19) United States
(12) Patent Application Publication
(10) Pub. No.: US 2023/0285557 A1
(43) Pub. Date: Sep. 14, 2023

(54) MULTI-PARALLEL ANALYSIS OF T-CELL THERAPIES

(71) Applicant: The Board of Trustees of the Leland Stanford Junior University, Stanford, CA (US)

(72) Inventors: Garry P. NOLAN, Palo Alto, CA (US); John W. HICKEY, Palo Alto, CA (US)

(21) Appl. No.: 18/004,636

(22) PCT Filed: Jul. 8, 2021
(86) PCT No.: PCT/US2021/040874

§ 371 Date: Jan. 6, 2023

Related U.S. Application Data
(60) Provisional application No. 63/049,943, filed on Jul. 9, 2020.

(51) Int. Cl. A61K 39/00; C12N 5/0783; G01N 33/574; A61P 35/00

(52) U.S. Cl. CPC A61K 39/4611; A61P 35/00; C12N 5/0636; G01N 33/57484; C12N 2501/999; C12N 2501/2302

(57) ABSTRACT

The present disclosure relates generally to ex vivo expanded T cell populations suitable for use in adoptive immunotherapy. The disclosure also provides compositions and methods useful for preparing such ex vivo expanded T cell populations, as well as methods for the prevention and/or treatment of health conditions using the disclosed T cell populations.

### STATEMENT REGARDING FEDERALLY SPONSORED R&D

This invention was made with Government support under contracts 2U19AI057229-16, 5P01HL10879707, 5R01GM10983604, 5R33CA18365403, 5U01AI101984-07, 5UH2AR06767604, 5R01CA19665703, 5U54CA20997103, 5F99CA212231-02, 1F32CA233203-01, 5U01AI140498-02, 1U54HG010426-01, 5U19AI100627-07, 1R01HL120724-01A1, R33CA183692, R01HL128173-04, 5P01AI131374-02, 5UG3DK114937-02, 1U19AI135976-01, IDIQ17X149, 1U2CCA233238-01, 1U2CCA233195-01 awarded by The National Institutes of Health. The Government has certain rights in the invention.

### CROSS-REFERENCE TO RELATED APPLICATIONS

This application claims the benefit of priority to U.S. Provisional Pat. Application Serial No. 63/049,943, filed on Jul. 9, 2020. The disclosure of the above-referenced application is herein expressly incorporated by reference it its entirety, including any drawings.

### FIELD

The present disclosure generally relates to the field of immunology, and particularly relates to ex vivo expanded T cell populations that are suitable for use in adoptive immunotherapy. The disclosure also provides compositions and methods useful for preparing such ex vivo expanded T cell populations, as well as methods for the prevention and/or treatment of health conditions using the disclosed T cell populations.

### BACKGROUND

Immune-cell based immunotherapy is a rapidly growing field that has experienced impressive clinical successes in the last few years. In particular, a wave of immune cell therapies has created an entirely new paradigm within cancer immunotherapy with substantial benefits from antigen-specificity, drug proliferation, and memory. For example, immune cell-based therapies such as T cell therapies have recently shown dramatic efficacy against cancers, including solid tumors and hematological malignancies. In particular, it is now possible to generate human T cells that display desired specificities and enhanced functionalities compared with the natural immune system. Ex vivo expansion and activation of T cells are pre-requisites for most forms of T cell immunotherapy. T cells, including recombinant both chimeric antigen receptor (CAR-T) and endogenous T cells, have been used therapeutically to achieve impressive clinical outcomes, with a majority of patients achieving complete remission. Similarly, natural killer (NK) cell therapies have proven clinical efficacy without needing secondary activation.

However, increasing utilization of T cell immunotherapies for treatment of malignancies and infectious diseases has been hindered by the lack of rapid, cost-effective, and efficient methods for selecting and expanding clinical-grade, therapeutic T cell products that proliferate and persist in vivo. In particular, cell therapies represent a particularly complex class of adaptive and “living” drugs. For example, it is difficult to produce clinically relevant doses of fully standardized products because of the heterogeneity of T cell phenotypes after ex vivo expansion. This is due to both a lack of control over phenotype in ex vivo cell culture and the difficulties associated with systematically and rigorously analyzing cell phenotype. In addition, many immune cells exhibit drastic plasticity and shift between tumor-promoting and tumor-killing roles. Thus, even if phenotype is controlled ex vivo, cell behavior could change in vivo and reduce therapeutic efficacy. Furthermore, once introduced into a receiving subject, a therapeutic cell will send and receive signals from cells other than its target. This causes unintended side effects that could be beneficial (e.g. immune cascade against tumor) or adverse (e.g. destruction of healthy tissue). Moreover, pharmacokinetics of living drugs are not straightforward because a) the cells administered can die, replace themselves, or multiply in unpredictable ways and b) cells may preferentially traffic to specific tissues based on their phenotype.

Accordingly, there remains a need in the art for more robust methodologies for expanding T cell populations with improved clinical therapeutic potential.

### SUMMARY

The present disclosure generally relates to improved compositions for adoptive cell therapy, and methods for making such compositions. Some embodiments of the disclosure provide methods for preparing ex vivo expanded T cell populations that are suitable for use in adoptive immunotherapy. In particular, some embodiments of the disclosure describe methods for preparing T cell populations in the presence of antigen stimulation, and an inhibitor of acetyl-CoA production, followed by characterization of the expanded T cells using a panel of biomarkers for T-cell specialization and/or exhaustion to determine if the expanded T cell populations is suitable for use in adoptive immunotherapy. The disclosure also provides ex vivo expanded T cell populations prepared by the methods of the disclosure, pharmaceutical compositions containing the same, and methods for the prevention and/or treatment of health conditions using the disclosed T cell populations and/or pharmaceutical compositions as described herein

In one aspect, provided herein are methods for preparing an ex vivo expanded T-cell population suitable for use in adoptive immunotherapy, the methods including: (a) expanding an input population of T cells in the presence of antigen stimulation and an inhibitor of acetyl-CoA production; (b) measuring the levels of a panel of biomarkers for T-cell specialization and/or exhaustion expressed in the expanded T-cell population to generate a cell composition profile; and (c) identifying the expanded T cell population as suitable for use in adoptive immunotherapy based at least in part upon the generated cell composition profile. In some embodiments, the methods further include the step of obtaining the input population of T cells from a subject.

Non-limiting exemplary embodiments of the disclosed methods can include one or more of the following features. In some embodiments, the inhibitor of acetyl-CoA production is an inhibitor of ATP-citrate lyase (ACL), acyl-CoA synthetase short-chain family member 2 (ACSS2), carnitine acetyltransferase (CAT), and/or or pyruvate dehydrogenase complex (PDC). In some embodiments, the ACL inhibitor is selected from the group consisting of hydroxycitric acid (HCA), bempedoic acid (ETC-1002), BMS-303141, SB 204990, and SB 201076, or a pharmaceutically acceptable salt thereof. In some embodiments, the ACL inhibitor comprises hydroxycitric acid (HCA) and/or a pharmaceutically acceptable salt thereof. In some embodiments, the HCA salt is tripotassium 2-hydroxycitrate (also referred to herein as 2-hydroxycitrate, “2HC”). In some embodiments, the 2HC concentration during expansion of the obtained T cells ranges from 1 mM to 100 mM.

In some embodiments, the panel of biomarkers for T-cell specialization and/or exhaustion comprises biomarkers representing one or more of the following features of the expanded T-cell population: proliferative potential, activated T cell, memory marker, further T cell differentiation, exhaustion, activation/memory/effector marker, stem cell memory marker, terminally differentiated marker, exhaustion marker, and T cell activation marker. In some embodiments, the panel of biomarkers for T-cell specialization and/or exhaustion comprises one or more of the following: CD3e, CD4, CD8, CD25, CD27, CD38, CD44, C62L, CD69, CD117, CD122, CD127, CD200r, CD279, CCR7, ICOS, KLRG1, Ly6C, Tim3, and Sca-1. In some embodiments, the cell composition profile includes relative proportions of the following cell subpopulations: Ly6C⁺ memory cells, Ly6C⁺/CD117⁺ memory cells, memory cells, exhausted effector cells, effector cells, Ly6C⁺ exhausted effector cells, terminally differentiated effector cells, Ly6C⁻ terminally differentiated effector cells, CD8- T cells, and CD38^{high}/CD27⁻ memory cells. In some embodiments, the generation of the cell composition profile comprises using biomarkers that delimit substantially the same population as Ly6C⁺ memory cells, Ly6C⁺/CD117⁺ memory cells, memory cells, exhausted effector cells, effector cells, Ly6C⁺ exhausted effector cells, terminally differentiated effector cells, Ly6C- terminally differentiated effector cells, CD8⁻ T cells, and CD3S^{high}/CD27⁻ memory cells. In some embodiments, the methods further include measuring levels of cytokines and/or effector molecules produced in the expanded T-cell population.

In some embodiments, the step of obtaining an input population of T cells further comprises introducing into the T cells an immune receptor and/or nucleic acids encoding the immune receptor. In some embodiments, the immune receptor is a T-cell receptor (TCR) or a chimeric antigen receptor (CAR).

In some embodiments, the step of measuring the levels of a panel of biomarkers comprises using a nucleic-acid-based analytical assay selected from the group consisting of nucleic acid amplification-based assays, polymerase chain reaction (PCR), real-time PCR, nucleic acid sequencing, quantitative reverse transcription PCR (qRT-PCR), PCR-RFLP assay, HPLC, mass-spectrometric genotyping, nucleic acid hybridization assay, comparative genomic hybridization, restriction digestion, capillary electrophoresis, and combinations of any thereof. In some embodiments, the step of measuring the levels of a panel of biomarkers comprises using a protein-based analytical assay selected from the group consisting of immunohistochemistry (IHC), protein-microarray, western blotting, mass spectrometry, flow cytometry, enzyme-linked immunosorbent assay (ELISA), immunofluorescence staining, multiplex detection assay, and combinations of any thereof.

In some embodiments, the methods disclosed herein further include harvesting the ex vivo expanded T cells. In some embodiments, the input population of T cells is obtained from a mammalian subject. In some embodiments, the subject has or is suspected of having a proliferative disorder, an autoimmune disorder, or an infection. In some embodiments, the proliferative disorder is a cancer.

In another aspect, provided herein are ex vivo expanded T-cell populations that are prepared by a method of the disclosure. Non-limiting exemplary embodiments of the ex vivo expanded T-cell populations as described herein can include one or more of the following features. In some embodiments, at least 60%, at least 70%, at least 80%, at least 90%, or at least 95% of the expanded T cells are memory T cells. In some embodiments, the memory T cells comprise central memory cells, stem cell memory cells, and effector memory cells. In some embodiments, the memory cells comprise an increased expression of one of more biomarkers selected from the group consisting of CD62L, CD127, CD44, CD95, CD27, and CCR7, compared to control cells that are not cultured in the presence of the inhibitor of acetyl-CoA production. In some embodiments, the ratio of memory T cells to effector T cells is about 2:1 to about 10:1. In some embodiments, the expanded T-cell population comprises a cell composition profile as set forth in Table 2 or Table 3. In some embodiments, the expanded T-cell population comprises one or more of the following characteristics: high proliferative capacity, self-renewing capacity, high activation state, high functionality/cytotoxicity, and low exhaustion profile.

In another aspect, provided herein are pharmaceutical compositions that include a T-cell population of the disclosure, and a pharmaceutically acceptable excipient.

In yet another aspect, provided herein are methods for preventing and/or treating a condition in a subject in need thereof, the method comprising administering to the subject a formulation comprising one or more of the following: (a) a T cell population of the disclosure; and/or (b) a pharmaceutical composition of the disclosure.

Non-limiting exemplary embodiments of the disclosed methods can include one or more of the following features. In some embodiments, the condition is a proliferative disorder, an autoimmune disorder, or an infection. In some embodiments, the T cells are autologous to the subject in need of treatment. In some embodiments, the T cells are allogeneic to the subject in need of treatment. In some embodiments, the subject has or is suspected of having a proliferative disorder, an autoimmune disorder, or an infection. In some embodiments, the proliferative disorder is a cancer. In some embodiments, the formulation is administered to the subject individually as a single therapy (monotherapy) or in combination with at least one additional therapies selected from the group consisting of chemotherapy, radiotherapy, immunotherapy, hormonal therapy, toxin therapy, targeted therapy, and surgery.

In another embodiments, provided herein are kits for the prevention and/or treatment of a heath condition in a subject in need thereof, the kit include: (a) a T cell population of the disclosure; and/or (b) a pharmaceutical composition of the disclosure.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1D graphically summarize the results of experiments performed to demonstrate that stimulation of murine PMEL T cells with and without 2HC leads to extreme phenotypes determined by CyTOF staining. FIG. 1A: Experimental diagram of murine T cell therapeutic model. FIG. 1B: Eleven major clusters from unsupervised clustering and UMAP projection of this data to 2D, in which PD1 expression and CD62L expression are shown to demonstrate that major clusters separate into effector, memory, and exhausted cell populations. FIG. 1C: Percentage of each cluster type (by color) for each condition. FIG. 1C: Trajectory analysis with minimal spanning tree reveals conditions skew to two major phenotypic nodes (# = cluster number, size = proportion of cells in cluster, color = expression of Tim3 as analyzed by “Cytometry by Time Of Flight Mass Spectrometry” (CyTOF).

FIGS. 2A-2E graphically summarize the imaging results from tumors taken on Day 3. FIG. 2A: CODEX (CO-Detection by indEXing) imaging of array of cancer sections on a slide was visualized by staining by a panel of DNA-barcoded antibodies (n=44 for this experiment) over n/3 cycles by fluorescent complimentary oligonucleotides. FIG. 2B: Imaging results of treated cancers on Day 3 with only 5 of 44 markers shown for clarity for each of the 3 different groups: No T cells, T cells, and T cells treated with 2HC that demonstrate differential ability of the cellular therapies to cause major microenvironmental changes to the tumor with MHC-I and immuno-inhibitory (PDL1) molecule expression. FIGS. 2C-2E: Percentages of different populations of T cells: CD45⁺, CD3⁺ cells (FIG. 2C); CD45⁺, CD3⁻ cells (FIG. 2D); and CD45⁻ cells (FIG. 2E).

FIGS. 3A-3F graphically summarize the results of experiments of cellular neighborhood analysis for CODEX imaging data. FIG. 3A: Neighborhood analysis revealed ten major cellular neighborhoods with distinct enrichments of certain cell types. FIG. 3B: Identified neighborhoods overlaid on original spatial coordinates with 7oronoi diagrams for each condition that show differential neighborhood quantities and also more conservation of neighborhoods in 2HC treated mice. FIG. 3C: Percent of each neighborhood type per each condition. FIGS. 3D-3F: Interaction analysis of the neighborhoods either (i) within the same neighborhood shown by neighborhood number (FIG. 3D) or condition (FIG. 3E); or between different neighborhoods along borders separated out by condition and plotted versus the interacting neighborhood (FIG. 3F).

FIG. 4 pictorially illustrates a model of how initial phenotype of T cells induces improved killing and anti-tumor responses and how that results in overall structure and eventual immune-tumor neighborhoods. In particular, 2HC-treated cells are able to establish pro-inflammatory supportive neighborhoods in the tumor while canonically treated T cells are not able to establish such neighborhoods resulting in sub-optimal therapeutic outcomes

FIG. 5 graphically illustrates a model for why phenotype of T cells causes additional anti-tumor effect and the concept of an initial inflammatory threshold.

FIGS. 6A-6C graphically summarize the results of experiments illustrating intracellular cytokine staining for T cells treated with and without 2HC as measured by CyTOF. FIG. 6A: 2HC cells secrete preferentially IL-7 and do so constitutively without need for stimulation and higher levels of IL-2 upon restimulation, whereas T cells treated without 2HC more effector molecules (perforin, granzyme B, and IFNγ) indicating a more differentiated cell. FIG. 6B: Comparing the amounts of different cytokine and effector molecules secreted by the same cell shows that T cells without 2HC have a higher percentage of cells that secrete multiple effector molecules. FIG. 6C: Zooming in with the phenotypic molecules used also within CyTOF, it was observed that T cells treated without 2HC that are secreting multiple effector molecules are also expressing cell exhaustion molecules like PD1 and Tim3 indicating late stage effector phenotype, whereas cytokine secreting cells in the 2HC condition are of memory phenotype.

FIG. 7 graphically summarize the results of RNA-seq experiments performed on T cells treated differentially (e.g., with or without 2HC). Genes represented in green/yellow (above the horizontal line) are enriched for 2HC-treated T cells, while genes represented in blue (below the horizontal line) are enriched in T cells not treated with 2HC. Gene categories presented in this figure include (i) phenotype-associated genes, (ii) cytokine-associated genes, (iii) IFNγ-associated genes, and (iv) migration-associated genes.

### DETAILED DESCRIPTION OF THE DISCLOSURE

The present disclosure generally relates to improved compositions for adoptive cell therapy, and methods for making such compositions. Some embodiments of the disclosure provide methods for preparing ex vivo expanded T cell populations that are suitable for use in adoptive immunotherapy. In particular, some embodiments of the disclosure describe methods for preparing T cell populations in the presence of antigen stimulation and an inhibitor of acetyl-CoA production, followed by characterization of the expanded T cells using a panel of biomarkers for T-cell specialization and/or exhaustion to determine if the expanded T cell populations is suitable for use in adoptive immunotherapy. The disclosure also provides ex vivo expanded T cell populations prepared by the methods of the disclosure, pharmaceutical compositions containing the same, and methods for the prevention and/or treatment of health conditions using the disclosed T cell populations and/or pharmaceutical compositions as described herein.

Immune-cell based immunotherapy is a rapidly growing field that has experienced impressive clinical successes in the last few years. For example, immune cell-based therapies such as T cell therapies have recently shown dramatic efficacy against cancers, including solid tumors and hematological malignancies. However, despite the great success of immune cell therapies, efficacy has been largely restricted to liquid tumors like chronic lymphocytic leukemia (CLL). Yet this only accounts for 5% of all cancer cases and deaths each year. Challenges observed in targeting solid cancers are: 1) low immune cell infiltration, 2) severe toxicity and adverse events such as cytokine release syndrome, 3) antigen/target loss, and 4) potent immunosuppressant cancer microenvironments.

Further complicating these cell therapies is the need for ex vivo manipulation of a patients cells to produce clinically relevant numbers (~10¹¹ per patient). This is challenging because it requires 5 to 7 weeks, which many patients may not have, many of the cells may become dysfunctional or exhausted outside the body for so long, the culture process is complex, technical, and non-standardized with a variety of cytokines, feeder cells, and antibodies used, and inability to perform quality control of ex vivo cell therapy phenotypes and cell states in multiplexed and comprehensive fashion, which limits ability to correlate function with ex vivo conditions.

Moreover, overall insight into what happens in vivo with these “living” drugs is very limited where many current assays dissociate cells for analyses. The spatial context is critical because (a) immune cells may be excluded from the TME16, (b) nearest tumor cells may not express specific antigen, and (c) knowing phenotypes of cellular neighbors will better predict synergistic therapies. This is highlighted by our lack of understanding of how the quality of the delivered, engineered immune cell “product” affects and is affected by immune/tumor networks and cell neighborhoods in the cancer microenvironment. In general, our understanding of the “push-pull” relationship between immune cell therapies and the cancer microenvironment has been limited, thus restricting our ability to design cell product therapies with greater efficacy.

Furthermore, it has been reported that immune cell phenotype is both dynamic and plastic, both in vivo and in vitro. As immune cell therapies require substantial ex vivo culture to expand therapeutic cells several thousand fold to get an adequate number of cells for the patient, this dynamic and plastic phenotype can lead to dramatic changes in cellular phenotype. In particular, features in these alien culture environments such as cytokine levels and types, amount of antigen, feeder cell presence, small molecule manipulations, and even salt concentration in culture media can have drastic implications in the phenotype.

Recent studies have revealed that certain immune cell phenotypes have been associated with better cancer killing outcomes and has been linked to immuno-inhibitory surface proteins, metabolic state, and cytokine expression. However, many of these studies have been isolated either in the (a) number of perturbations to immune cells, (b) combination of different type of perturbations (cytokines vs. metabolic inhibitors, etc.), (c) number of markers used to evaluate the phenotypic space, or (d) the use of both in vitro and in vivo efficacy models. This has limited to the insight to the entire phenotypic space, plasticity, and association with function of these therapies. Indeed novel nuanced cell phenotypes are still continually being discovered.

It is not entirely clear how phenotype leads to changes in therapeutic efficacy in immune cell therapies. In fact, some studies over cellular phenotype and in vivo function have been contradictory. This is further complicated by the observation that cellular phenotypes characterized by only a couple of markers (e.g., CD44, CD62L) may not fully represent the phenotypic diversity actually present in cultures. Furthermore, with two FDA T cell therapies recently approved, very little is known about the “push-pull” relationship of cancer-specific T cells and the cancer microenvironment.

There is increasing evidence that the organization of immune cells and cancer cells is linked with survival. Particularly, spatially concentrated T cells are believed to be important to increases in survival. Some recent studies have suggested that antigen-specific responses may be drivers of tertiary lymphoid structure formation. While T cell spatial relationship in the cancer microenvironment has been studied, little has been done to look at their neighbors, associations, and interactions in-depth and simultaneously. Additionally, the quality of the immune cell product has dramatic effects on anti-cancer responses, but little has been studied in terms of how the quality of immune cell therapies affect the cancer microenvironment neighborhoods and structure. Understanding both are important to designing more effective therapies in the future.

As described in greater detail herein, the present disclosure provides a standardized, multi-parallel, and high-dimensional system for investigating effects of ex vivo cell manipulation and their mechanistic and therapeutic impacts in vivo for solid tumors. In particular, the biologic insight of how ex vivo cell phenotype relates to therapeutic efficacy will springboard the community’s ability to engineer and redesign more effective cell therapies going forward or other druggable targets. Uniquely, the studies presented herein employ both single-cell and image-based multi-cell analyses which facilitate the identification of spatial and temporal limitations and mechanisms of cell therapies. Additionally, these studies also reveal additional biomarkers that can be used to predict patient response to these therapeutics. Without being bound to any particular theory, the approach described herein is applicable across cell therapies and targets.

Using these approaches, the experimental data presented herein demonstrates that T cells can be controlled in a state that enable complete transformation of tumor cell internal growth program, overcoming tumor immunosuppressive factors, and increased killing of tumor cells. Since the T cell phenotype was the only variable in these studies, this finding indicates that there is some factor or combination of factors released by transferred T cells or other recruited cells because of the T cell treatment that caused the changes in the tumor growth program. This finding is novel as primarily T cell therapies are thought to have only killing mechanisms, rather than anti-proliferative effects. Therefore, this finding is unique not only in controlling T cell fate, but as it is understood mechanistically it could result in drugs which cause the same effect.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “administration” and “administering”, as used herein, refer to the delivery of a bioactive composition or formulation by an administration route comprising, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

As used herein, a “subject” or an “individual” includes animals, such as human (e.g., human subjects) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” As discussed above, the term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is understood that aspects and embodiments of the disclosure described herein include “comprising,” “consisting,” and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub- combination was individually and explicitly disclosed herein.

### Acetyl-Coa Production in T Cells and Metabolic Control of T Cell Memory

Acetyl-CoA is a central molecule in cell metabolism, signaling, and epigenetics. It serves crucial roles in energy production, macromolecular biosynthesis, and protein modification. It has been reported that cellular levels of acetyl-CoA affects immune, cancer, and stem cell functions. In particular, in highly proliferative cells such as T cells, increased cytosolic acetyl-CoA levels are required for histone acetylation to promote interferon-γ (INFγ) production. Depletion of nucleo-cytosolic acetyl-CoA limits the acquisition of histone acetylation on the promoters and enhancers of genes encoding effector molecules. Simultaneously, nutrient deprivation reduced methionine intermediates, depressing methylation of histone marks that normally suppress stemness-associated programs. Treatment of antitumor T cells with elevated extracellular potassium as well as pharmacologic or gene therapies mimicking mechanisms of functional starvation resulted in T cells with retained stemness, evidenced by self-renewal and multipotency, thereby enabling the enhanced destruction of large, established tumors.

Within mitochondria, acetyl-CoA is generated from pyruvate by the pyruvate dehydrogenase complex (PDC), as well as from catabolism of fatty acids and amino acids. To enter the TCA cycle, acetyl-CoA condenses with oxaloacetate, producing citrate, a reaction catalyzed by citrate synthase. Transfer of acetyl-CoA from mitochondria to the cytosol and nucleus involves the export of citrate and its subsequent cleavage by ATP-citrate lyase (ACL), generating acetyl-CoA and oxaloacetate. This acetyl-CoA is used for a number of important metabolic functions, including synthesis of fatty acids, cholesterol, and nucleotide sugars such as UDP-N-acetylglucosamine. Acetyl-CoA also serves as the acetyl-group donor for both lysine and N-terminal acetylation.

In addition to ACL, nuclear-cytosolic acetyl-CoA can also be produced from acetate by acyl-CoA synthetase short chain family member 2 (ACSS2). Two additional acetyl-CoA-producing enzymes, the PDC and carnitine acetyltransferase (CAT), have been reported to be present in the nucleus and to contribute to acetyl-CoA production. In addition, the PDC was shown to translocate from mitochondria to the nucleus under certain conditions, such as growth factor stimulation; within the nucleus, the complex is intact and retains the ability to convert pyruvate to acetyl-CoA.

It has been reported that the mitochondrial requirements are diverse among different cell types and they highly influenced by the microenvironment. To meet the increased energy requirement associated with cell division and effector function, primed T cells undergo a metabolic switch from oxidative phosphorylation to aerobic glycolysis. For optimal function upon restimulation, memory CD8⁺ T cells must increase their aerobic metabolic rate to promote the production of citrate, which can be converted to acetyl-CoA outside of the mitochondrion and may be used as a substrate by histone acetyltransferases. As memory T cells are functionally defined by their ability to respond more quickly to restimulation than naive cells, memory cells may adapt their metabolism to support a rapid response. Furthermore, the rapid effector response by restimulated memory CD8⁺ T cells is supported by an increased respiratory rate, which promotes the production of extra-mitochondrial acetyl-CoA downstream of the tricarboxylic acid (TCA) cycle.

### Methods for Preparing Ex Vivo Expanded T-Cell Population

As described in greater detail below, one aspect of the present disclosure relates to methods for preparing an ex vivo expanded T-cell population suitable for use in adoptive immunotherapy. In some embodiments, the methods described herein include: (a) expanding an input population of T cells in the presence of antigen stimulation and an inhibitor of acetyl-CoA production; (b) measuring the levels of a panel of biomarkers for T-cell specialization and/or exhaustion expressed in the expanded T-cell population to generate a cell composition profile; and (c) identifying the expanded T cell population as suitable for use in adoptive immunotherapy based on the generated cell profile, as indicated by a high proportion of memory T cells in the expanded T-cell population. In some embodiments, the methods further include the step of obtaining the input population of T cells from a subject.

### Input T Cells

Non-limiting exemplary embodiments of the disclosed methods can include one or more of the following features. In some embodiments, the input population of T cells is isolated or purified from a mammal, e.g., a human. In principle, the input population of T cells according some embodiments of the disclosure can include one or more types of T cells. Generally, the input T cells can be any types of T cells. For example, input T cells can include a cultured T cell, e.g., a primary T cell, or a T cell from a cultured T cell line, e.g., Jurkat, SupT1, or Molt3. In instances where input T cells are obtained from a mammal, the input T cells can be obtained from any suitable sources, including but not limited to blood, bone marrow, lymph node, the thymus, tumor, or other tissues or fluids. Input cells can be isolated by any suitable method known in the art. For example, input cells can be obtained from the mammal by a blood draw or a leukapheresis. In some embodiments, the cells include peripheral blood mononuclear cells (PBMC). In some embodiments, input T cells are optionally enriched or purified prior to the expansion step. In principle, input T cells can be of any developmental stage, including but not limited to, CD4⁺/CD8⁺ double positive T cells, CD4⁺ T cells, e.g., Thi and Th2 cells, CD8⁺ T cells (e.g., cytotoxic T cells), Th9 cells, memory T cells, naive T cells, and the like. In some embodiments, input T cells include at least one CD8⁺ T cell or CD4⁺ T cell. Alternatively or additionally, input T cells can be obtained from a tumor sample taken from the mammal. For example, in some embodiments, input T cells include tumor infiltrating lymphocytes (TIL).

In some embodiments, the step of obtaining an input population of T cells includes introducing into the T cells an immune receptor, such as a T-cell receptor (TCR or a chimeric antigen receptor (CAR), and/or nucleic acids encoding the same. For example, the input T cells can include and/or express an antigen-specific receptor, e.g., a receptor that can immunologically recognize and/or specifically bind to an antigen, or an epitope thereof, such that binding of the antigen-specific receptor to antigen, or the epitope thereof, elicits an immune response. In some embodiments, the antigen-specific receptor has antigenic specificity for a cancer antigen, such as a tumor-specific antigen (TSA) or a tumor-associated antigen (TAA).

In some embodiments, the antigen-specific receptor is a T-cell receptor (TCR). A TCR generally comprises two polypeptides (e.g., polypeptide chains), such as an α-chain of a TCR, a β-chain of a TCR, a γ-chain of a TCR, a γ-chain of a TCR, or a combination thereof. Such polypeptide chains of TCRs are known in the art. The antigen-specific TCR can include any amino acid sequence, provided that the TCR can specifically bind to and/or immunologically recognize an antigen, such as a cancer antigen or epitope thereof. In some embodiments, the TCR is an endogenous TCR, e.g., a TCR that is endogenous or native to (naturally-occurring) the T cell. In such a case, the T cell expressing the endogenous TCR can be a T cell that was isolated from a mammal which is known to express the particular cancer antigen. For example, in some embodiments, the T cell is a primary T cell isolated from a mammal having a cancer. In some embodiments, the T cell is a TII, or a T cell isolated from a human cancer patient.

In some embodiments, the input T cells include and/or express a chimeric antigen receptor (CAR). Generally, a CAR includes an antigen binding domain, e.g., a single-chain variable fragment (scFv) of an antibody, fused to a transmembrane domain and an intracellular domain. In this case, the antigenic specificity of a CAR can be encoded by a scFv which specifically binds to the antigen, or an epitope thereof. CARs, and methods of making them, are known in the art.

In some embodiments, the input T cells include one or more nucleic acids encoding an exogenous (e.g., recombinant) antigen-specific receptor. In some embodiments, such exogenous antigen-specific receptors, e.g., exogenous TCRs and CARs can confer specificity for additional antigens to the T cell beyond the antigens for which the endogenous TCR is naturally specific.

### Ex Vivo Expansion of T Cells

As described in greater detail below, the input T cells are cultured or expanded in the presence of an inhibitor of acetyl-CoA production in the cells. In some embodiments, the inhibitor of acetyl-CoA production is an inhibitor of ATP-citrate lyase (ACL), acyl-CoA synthetase short-chain family member 2 (ACSS2), carnitine acetyltransferase (CAT), and/or or pyruvate dehydrogenase complex (PDC).

In some embodiments, the input T cells are cultured or expanded in the presence of an inhibitor of ACL activity. Suitable inhibitors of ACL activity include, but are not limited to 3,3,14,14-Tetramethylhexadecanedioic acid (CAS Number 87272-20-6; NDI-091143), hydroxycitric acid (HCA) and salts thereof (e.g., pharmaceutically acceptable salts thereof), bempedoic acid (ETC-1002) and salts thereof (e.g., pharmaceutically acceptable salts thereof), BMS-303141, SB 204990, and SB 201076, or a salt thereof (e.g., pharmaceutically acceptable salt thereof). Additional ACL inhibitors suitable for the compositions and methods disclosed herein include, but are not limited to those described in, for example, Granchi C., Eur. J. Medicinal Chemistry, Vol. 157, 5 Sept. 2018, pp. 1276-1291. In some embodiments, the input T cells are cultured or expanded in the presence of hydroxycitric acid (HCA) and/or a salt thereof (e.g., pharmaceutically acceptable salt thereof). In some embodiments, the HCA salt is potassium hydroxycitrate or sodium hydroxycitrate. In some embodiments, the HCA salt is a hydrate thereof. In some embodiments, the HCA salt is tripotassium 2-hydroxycitrate (e.g., potassium hydroxycitrate tribasic monohydrate; “2HC”).

Exemplary inhibitors of acyl-CoA synthetase short-chain family member 2 (ACSS2) suitable for the methods and compositions of the disclosure include (N-(2,3-di-2-thienyl-6-quinoxalinyl)-N -(2-methoxyethyl)urea (CAS 508186-14-9) and those described in PCT Patent Publication Nos. WO2019067528A1, and WO2019097515A1. Suitable inhibitors of carnitine acetyltransferase (CAT) for the methods and compositions of the disclosure include, but are not limited to methoxycarbonyl-CoA disulfide, hemiacetylcarnitinium (HAC), acetyl-DL-aminocarnitine, allicin, and bromoacetyl carnitine. Additional CAT inhibitors suitable for the compositions and methods disclosed herein include, but are not limited to those described in, for example, Colucci W.J. et al., Bioorganic Chemistry, Vol. 16, Issue 3, September 1988, pp. 307-334. Exemplary inhibitors of pyruvate dehydrogenase complex (PDC) suitable for the methods and compositions of the disclosure include VER-246608, AZD7545, thiamin thiazolone diphosphate, methylacetyl phosphonate, and those described in PCT Patent Publication No. WO2015040424A1. Additional PDC inhibitors suitable for the compositions and methods disclosed herein include, but are not limited to those described in, for example, Stacpoole P.W., Journal of the National Cancer Institute, Vol. 109, Issue 11, November 2017.

In some embodiments, the inhibitor of acetyl-CoA production is present in the culture at a concentration ranging from about 1 mM to about 100 mM, such as, for example, about 1 mM to about 50 mM, about 5 mM to about 70 mM, about 10 mM to about 80 mM, about 20 mM to about 90 mM, about 30 mM to about 100 mM, about 1 mM to about 20 mM, about 2 mM to about 15 mM, about 3 mM to about 10 mM, about 5 mM to about 20 mM, about 10 mM to about 15 mM, about 15 mM to about 20 mM, or about 1 mM to about 10 mM. In some embodiments, the inhibitor of acetyl-CoA production is present in the culture at a concentration ranging from about 1 mM to about 10 mM, for example, about 1 mM, about 2 mM, about 3 mM, about 4 mM, about 5 mM, about 6 mM, about 7 mM, about 8 mM, about 9 mM, or about 10 mM. In some embodiments, the inhibitor of acetyl-CoA production is present in the culture at a concentration of about 5 mM.

In some embodiments, the cells can be cultured ex vivo in the presence of an inhibitor of acetyl-CoA production intermittently. In some embodiments, the cells are cultured in the presence of an inhibitor of acetyl-CoA production for the entire duration of ex vivo culture, including during expansion of the numbers of cells and during any introduction of a nucleic acid encoding an antigen-specific TCR or CAR into the cells.

In some embodiments, the cell culture medium can further include any of a variety of additives. For example, the cell culture medium may further comprise one or more antibodies and/or one or more cytokines. Accordingly, in some embodiments, the expansion step of the methods described herein include culturing the cells in the presence of (a) an inhibitor of the acetyl-CoA production and (b) a cytokine such as, for example, interleukin-2 (IL-2), interleukin-7 (IL-7), interleukin-15 (IL-15), interleukin-12 (IL- 12) or a combination of two or more of the foregoing cytokines. In some embodiments, the expansion step of the methods described herein include culturing the cells in the presence of an inhibitor of the acetyl-CoA production and IL-2.

In some embodiments, the expanded T cells exhibit antigenic specificity for an antigen, for example a cancer antigen. The term “cancer antigen,” as used herein, refers to any molecule (e.g., protein, polypeptide, peptide, lipid, carbohydrate, etc.) solely or predominantly expressed or over-expressed by a tumor cell or cancer cell, such that the antigen is associated with the tumor or cancer. The cancer antigen can additionally be expressed by normal, non-tumor, or non-cancerous cells. However, in such cases, the expression of the cancer antigen by normal, non-tumor, or non-cancerous cells is not as robust as the expression by tumor or cancer cells. In this regard, the tumor or cancer cells can over-express the antigen or express the antigen at a significantly higher level, as compared to the expression of the antigen by normal, non-tumor, or non-cancerous cells. Also, the cancer antigen can additionally be expressed by cells of a different state of development or maturation. For instance, the cancer antigen can be additionally expressed by cells of the embryonic or fetal stage, which cells are not normally found in an adult host. Alternatively, the cancer antigen can be additionally expressed by stem cells or precursor cells, which cells are not normally found in an adult host.

The cancer antigen can be an antigen expressed by any cell of any cancer or tumor, including the cancers and tumors described herein. The cancer antigen may be a cancer antigen of only one type of cancer or tumor, such that the cancer antigen is associated with or characteristic of only one type of cancer or tumor. Alternatively, the cancer antigen may be a cancer antigen (e.g., may be characteristic) of more than one type of cancer or tumor. For example, the cancer antigen may be expressed by both breast and prostate cancer cells and not expressed at all by normal, non-tumor, or non-cancer cells. Cancer antigens are known in the art and include, for example, CXorf6l, mesothelin, CD 19, CD22, CD276 (B7H3), gplOO, MART-l, Epidermal Growth Factor Receptor Variant III (EGFRVIII), TRP- 1, TRP-2, tyrosinase, NY-ESO-l (also known as CAG-3), MAGE-l, MAGE-3, etc.

In some embodiments, the T cells may include and express an antigen-specific receptor. One skilled in the art will understand that the phrases “antigen-specific” and “antigenic specificity,” generally mean that antigen-specific receptor can specifically bind to and immunologically recognize an antigen, or an epitope thereof, such that binding of the antigen-specific receptor to antigen, or the epitope thereof, elicits an immune response. In some embodiments, the antigen-specific receptor has antigenic specificity for a cancer antigen (also termed a tumor antigen or a tumor-associated antigen).

### High-Throughput Profiling of Ex Vivo Expanded T Cells

As described above, for many reasons, cell therapies have resulted in severe adverse events for a subset of patients and have limited efficacy in solid tumors. Consequently, there is a need for approaches and technologies that can systematically analyze cell therapies produced ex vivo and their effects in vivo. As described above, after ex vivo expansion and activation, the ex vivo expanded T cells are evaluated by measuring the levels of a panel of biomarkers for T-cell specialization and/or exhaustion expressed in the expanded T-cell populations to generate a cell composition profile.

In some embodiments, the ex vivo expanded T cells are evaluated by measuring the levels of biomarkers representing the following features: proliferative potential (e.g., Sca-1), activated T cell (e.g., CD25, D38, and CD69), memory marker (e.g., CD27, CD127, and CD62L), T cell (e.g., CD3e and CD8), further T cell differentiation (e.g., Ly6C), memory marker (e.g., CD127 and CD62L), exhaustion (e.g., CD279), activation/memory/effector marker (e.g., CD44), stem cell memory marker (e.g., CD117), terminally differentiated marker (e.g., KLG1), exhaustion marker (e.g., Tim3), and T cell activation marker (e.g., ICOS).

In some embodiments, the panel of biomarkers for T-cell specialization and/or exhaustion comprises one or more of the following: CD3e, CD4, CD8, CD25, CD27, CD38, CD44, C62L, CD69, CD117, CD122, CD127, CD200r, CD279, CCR7, ICOS, KLRG1, Ly6C, Tim3, and Sca-1, to generate a cell composition profile of the ex vivo expanded T cell populations.

In some embodiments, the levels of the panel of biomarkers are determined by using a CyTOF technique (Cytometry by Time Of Flight mass spectrometry) which employ antibodies conjugated to metal isotope tags to analyze more than 40 targets simultaneously in single cells. In addition, in some embodiments, a CODEX imaging technique (CO-Detection by indEXing) is used to extensively profile immune cell phenotypes following ex vivo manipulations and leverage CODEX imaging to understand the dynamic, spatial, in vivo tumor and immune responses to these therapeutics. The combination of CyTOF and CODEX allows: a) deliver greater biological insight to phenotypic diversity and plasticity of cells used in therapies, b) use these in-depth phenotypic profiles to increase control over cell phenotypes within many different ex vivo environments and manipulations, c) provide a unique single-cell resolution perspective into in vivo spatial systemic effects, salient cell-cell interactions, and sustained phenotype of cell therapies, and d) use the combined ex vivo and in vivo mechanistic data to rationally design cell therapies to be more effective in solid tumors. In some embodiments, the methods described herein further include deployment of various bioinformatics tools for computational analysis of both multi-parameter single-cell datasets including clustering, pseudo-time, and spatial neighborhood analysis.

In some embodiments, the cell composition profile includes relative proportions of the following cell subpopulations in the CD8⁺ T cells: (1) central memory, (2) stem cell memory, (3) effector memory, (4) naive, (5) effector, (6) terminally differentiated, and (7) exhausted (see, also Example 1 and Tables 1-3). In some embodiments, the T cell populations prepared in accordance with a method described herein contain a much higher proportion of memory like CD8⁺ T cells compared to a control T-cell population. In some embodiments, the control T-cell population includes cells identical to the cells cultured in the presence of the inhibitor(s) of acetyl-CoA production except that the control cells are not cultured in the presence of the inhibitor(s) of acetyl-CoA production. In some embodiments, the control T cell population contains a much higher proportion of effector and exhausted phenotypes (see, e.g., Example 1).

In some embodiments, the cell composition profile includes relative proportions of the following cell subpopulations: Ly6C⁺ memory cells, Ly6C⁺/CD117⁺ memory cells, memory cells, exhausted effector cells, effector cells, Ly6C⁺ exhausted effector cells, terminally differentiated effector cells, Ly6C⁻ terminally differentiated effector cells, CD8⁻ T cells, and CD38 ^{high}/CD27⁻ memory cells. In some embodiments, the generation of the cell composition profile comprises using biomarkers that delimit substantially the same population as Ly6C⁺ memory cells, Ly6C⁺/CD117⁺ memory cells, memory cells, exhausted effector cells, effector cells, Ly6C⁺ exhausted effector cells, terminally differentiated effector cells, Ly6C⁻ terminally differentiated effector cells, CD8⁻ T cells, and CD38^{high}/CD27⁻ memory cells.

In some embodiments, the methods further include measuring levels of one or more cytokines and/or effector molecules produced in the expanded T-cell population. In some embodiments, the one or more cytokines include IL-2 and/or IL-7. In some embodiments, the one or more effector molecules include perforin, granzyme B, and IFNy.

In the methods described herein, the step of measuring levels of biomarkers can be performed using one or more nucleic-acid-based analytical assays, protein-based analytical assay, or a combination thereof. Non-limiting examples of detection reagents suitable for the methods and systems of the disclosure include single-stranded nucleic acids (e.g., primers, probes), double-stranded nucleic acids, non-fluorescent and fluorescent nucleic acid-specific dyes, enzymes, and antibodies.

In some embodiments, the step of measuring the levels of a panel of biomarkers comprises using a nucleic-acid-based analytical assay selected from the group consisting of nucleic acid amplification-based assays, polymerase chain reaction (PCR), real-time PCR, nucleic acid sequencing, quantitative reverse transcription PCR (qRT-PCR), PCR-RFLP assay, HPLC, mass-spectrometric genotyping, nucleic acid hybridization assay, comparative genomic hybridization, restriction digestion, capillary electrophoresis, and combinations of any thereof. In some embodiments, the step of measuring the levels of a panel of biomarkers comprises using a protein-based analytical assay selected from the group consisting of immunohistochemistry (IHC), protein-microarray, western blotting, mass spectrometry, flow cytometry, enzyme-linked immunosorbent assay (ELISA), immunofluorescence staining, multiplex detection assay, and combinations of any thereof. Additional assays suitable for the methods disclosed herein include single-cell RNA sequencing, single-cell ATAC sequencing, bulk RNA sequencing, metabolomics, proteomics, and multiplexed imaging such as CO-Detection by indEXing (CODEX).

In some embodiments, the methods disclosed herein further include harvesting the ex vivo expanded T cells.

In some embodiments, the input population of T cells is obtained from a mammalian subject. In some embodiments, the subject has or is suspected of having a proliferative disorder, an autoimmune disorder, or an infection. In some embodiments, the proliferative disorder is a cancer.

Without being bound to any particular theory, in some embodiments, ex vivo expanded populations of T cells considered to be suitable for use in adoptive cell therapies if they contain high proportions of memory like T cells and/or low proportions of effector T cells and exhausted T cells. For example, ex vivo expanded populations of T cells considered to be suitable for use in adoptive cell therapies are those having one or more of the following features: (i) high proliferative capacity characterized by memory markers such as, e.g., CD122, CD27, CCR7, and CD127; (2) self-renewing capacity characterized by stem cell markers such as TCF7, Sca-1, CD117, and D62L; (3) high activation state characterized by markers such as CD69, CD44, ICOS, CD25, and CD38; (4) high functionality/cytotoxicity characterized by cytokines such as IFNγ, TNFα, IL-2, CD95, and degranulation markers such as CD107a, perforin, granzyme b; and (5) low exhaustion profile characterized by low PD1, Tim3, KLRG1. (See, e.g., Table 3).

### Ex Vivo Expanded T-Cell Population and Pharmaceutical Compositions

### Ex Vivo Expanded T Cell Populations

As described in greater detail below, the disclosure also provides populations of ex vivo expanded T cells that are suitable for use in adoptive immunotherapies. In some embodiments, the ex vivo expanded T cells are prepared by a method of the disclosure. For examples, in some embodiments, provided herein are T cell populations that are expanded ex vivo in the presence of antigen activation and one or more inhibitors of acetyl-CoA production. Non-limiting exemplary embodiments of the ex vivo expanded T-cell populations as described herein can include one or more of the following features. In some embodiments, at least 60% of the cells in the T cell populations described herein are memory T cells (e.g., memory like CD8⁺ T cells). For example, in some embodiments, at least 65%, at least 70%, at least 75%, at least 80%, at least 85%, at least 90%, or at least 95% of the cells in the ex vivo expanded T cell populations of the disclosure are memory T cells. In some embodiments, the memory T cells comprise central memory cells, stem cell memory cells, and effector memory cells.

In some embodiments, the memory cells comprise an increased expression of one of more biomarkers selected from the group consisting of CD62L, CD127, CD44, CD95, CD27, and CCR7, compared to control cells that are not cultured in the presence of the inhibitor of acetyl-CoA production. In some embodiments, the central memory cells display reduced expression level of CD95, and increased expression levels of CD62L, CD127, CD44, CD27, and CCR7. In some embodiments, the central memory cells display reduced expression level of CD44, and increased expression levels of CD62L, CD127, CD27, CD95, and CCR7. In some embodiments, the ratio of memory T cells to effector and/or exhausted T cells is at least about 2:1, for example, at least about 2:1, at least about 3:1, at least about 4:1, at least about 5:1, at least about 6:1, at least about 7:1, at least about 8:1, at least about 9:1, or at least about 10:1. In some embodiments, the ratio of memory T cells to effector and/or exhausted T cells ranges from about 2:1 to about 10:1, for example, from about 2:1 to about 10:1, from about 2:1 to about 10:1, from about 2:1 to about 10:1, from about 2:1 to about 8:1, from about 3:1 to about 10:1, from about 3:1 to about 8:1, from about 4:1 to about 10:1, from about 4:1 to about 8:1, from about 2:1 to about 5:1, or from about 4:1 to about 6:1. In some embodiments, the expanded T-cell population comprises a cell composition profile as set forth in Table 2 or Table 3.

In some embodiments, the ex vivo expanded T cell populations of the disclosure include one or more of the following properties: (i) high proliferative capacity characterized by memory markers such as e.g. CD122, CD27, CCR7, and CD127; (2) self-renewing capacity characterized by stem cell markers such as TCF7, Sca-1, CD117, and D62L; (3) high activation state characterized by markers such as CD69, CD44, ICOS, CD25, and CD38; (4) high functionality/cytotoxicity characterized by cytokines such as IFNy, TNFα, IL-2, CD95, and degranulation markers such as CD107a, perforin, granzyme b; and (5) low exhaustion profile characterized by low PD1, Tim3, KLRG1. In some embodiments, the ex vivo expanded T cell populations of the disclosure has a cell composition profile as described in Table 3. In some embodiments, the ex vivo expanded T cell populations of the disclosure has a cell composition profile as described in Table 2.

### Pharmaceutical Compositions

The ex vivo expanded T-cell populations and pharmaceutical compositions of the disclosure can be incorporated into compositions, including pharmaceutical compositions. Such compositions generally include one or more T-cell populations as described herein and a pharmaceutically acceptable excipient, e.g., carrier. Accordingly, in one aspect, some embodiments of the disclosure relate to pharmaceutical compositions for treating, preventing, ameliorating, reducing or delaying the onset or recurrence of a health condition, for example a proliferative disease (e.g., cancer), or a symptom thereof. In some embodiments, a pharmaceutical composition of the disclosure includes an ex vivo expanded T-cell population of the disclosure, and a pharmaceutically acceptable excipient.

In some embodiments, the pharmaceutical compositions disclosed herein include T-cell cultures that can be washed, treated, combined, supplemented, or otherwise altered prior to administration to a subject in need thereof. Furthermore, administration can be at varied doses, time intervals or in multiple administrations.

The pharmaceutical compositions provided herein can be in any form that allows for the composition to be administered to a subject. In some specific embodiments, the pharmaceutical compositions are suitable for mammalian administration, e.g., human administration. One of ordinary skill in the art will appreciate that the term “pharmaceutically acceptable” generally refers to substances approved by a regulatory agency of the Federal or a state government or listed in the U.S. Pharmacopeia or other generally recognized pharmacopeia for use in animals, and more particularly in humans. For example, the term “pharmaceutically acceptable excipient” as used herein generally refers to any suitable substance that provides a pharmaceutically acceptable carrier, additive, or diluent for administration of a compound or substance of interest into a subject. As such, “pharmaceutically acceptable excipient” can encompass substances referred to as pharmaceutically acceptable diluents, pharmaceutically acceptable additives, and pharmaceutically acceptable carriers. Suitable “pharmaceutically acceptable carrier” includes, but is not limited to, saline, solvents, dispersion media, coatings, antibacterial agents and antifungal agents, isotonic and absorption delaying agents, and the like, compatible with pharmaceutical administration. Supplementary active compounds (e.g., antibiotics and additional therapeutic agents) can also be incorporated into the compositions.

The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol aa, talc, sodium chloride, dried skim milk, glycerol, propylene, glycol, water, ethanol and the like. Additional examples of suitable pharmaceutical carriers are described in “Remington’s Pharmaceutical Sciences” by E.W. Martin. In some embodiments, the pharmaceutical composition of the disclosure is sterilely formulated for administration into a subject. In some embodiments, the individual is a human. One of ordinary skilled in the art will appreciate that the formulation should suit the mode of administration.

In some embodiments, the pharmaceutical compositions of the present disclosure are formulated to be suitable for the intended route of administration to a subject. For example, the pharmaceutical composition may be formulated to be suitable for parenteral, intraperitoneal, colorectal, intraperitoneal, and intratumoral administration. In some embodiments, the pharmaceutical composition may be formulated for intravenous, oral, intraperitoneal, intratracheal, subcutaneous, intramuscular, topical, or intratumoral administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### Methods and Kits for the Treatment of Health Conditions

Administration of any one of the therapeutic compositions described herein, e.g., ex vivo expanded T-cell populations and pharmaceutical compositions, can be used to treat patients in the treatment of relevant health conditions, such as proliferative diseases (e.g., cancer), autoimmune diseases, and chronic infections. In some embodiments, one or more T-cell populations and pharmaceutical compositions as described herein can be incorporated into therapeutic agents for use in methods of treating a subject who has, who is suspected of having, or who may be at high risk for developing one or more health conditions, such as proliferative diseases (e.g., autoimmune diseases, cancers) and chronic infections. In some embodiments, the individual is a patient under the care of a physician.

Accordingly, in one aspect, some embodiments of the disclosure relate to methods for preventing and/or treating a condition in a subject in need thereof, the method comprising administering to the subject a formulation comprising one or more of the following: (a) a T cell population of the disclosure; and/or (b) a pharmaceutical composition of the disclosure.

In some embodiments, the methods include administering a therapeutically effective amount of a T-cell population and/or pharmaceutical composition of the disclosure to a subject in need thereof. The term “effective amount”, “therapeutically effective amount”, or “pharmaceutically effective amount” of a subject T-cell population or composition of the disclosure generally refers to an amount or number sufficient for a T-cell population or a composition to accomplish a stated purpose relative to the absence of the T-cell population or composition (e.g., achieve the effect for which it is administered, treat a disease, reduce a signaling pathway, or reduce one or more symptoms of a disease or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a T-cell population or composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

In a non-limiting exemplary workflow, a method of the disclosure involves culturing input T cells ex vivo in the presence of an inhibitor of acetyl-CoA production in the cells; administering the ex vivo cultured T cells to a mammalian subject after culturing the cells in the presence of antigen stimulation and an inhibitor of acetyl-CoA production. The input population of T cells can be cultured ex vivo in the presence of inhibitor of acetyl-CoA production. After ex vivo expansion, the cultured cells are evaluated by measuring the levels of a panel of biomarkers for T-cell specialization and/or exhaustion expressed in the expanded T-cell population to generate a cell composition profile, and then transferred into a mammal affected by a health condition, such as cancer. Such a cell transfer method is generally referred to in the art as “adoptive cell transfer” or “adoptive cell therapy” (ACT). In some embodiments, the inhibitor of acetyl-CoA production is removed (e.g., washed) from the cell culture prior to administering the cells to the mammal subject. In some embodiments, the inhibitor of acetyl-CoA production is not removed from the cells prior to administering the cells to the mammal subject. In some embodiments, the methods described herein include administering a pharmaceutical composition comprising the ex vivo expanded T cells to the mammal subject.

Exemplary proliferative diseases can include, without limitation, autoimmune diseases, angiogenic diseases, a metastatic diseases, tumorigenic diseases, neoplastic diseases and cancers. In some embodiments, the proliferative disease is a cancer. The term “cancer” generally refers to a disease characterized by the rapid and uncontrolled growth of aberrant cells. The aberrant cells may form solid tumors or constitute a hematological malignancy. Cancer cells can spread locally or through the bloodstream and lymphatic system to other parts of the body. There are no specific limitations with respect to the cancers which can be treated by the compositions and methods of the present disclosure. Non-limiting examples of suitable cancers include ovarian cancer, renal cancer, breast cancer, prostate cancer, liver cancer, brain cancer, lymphoma, leukemia, cervical cancer, skin cancer, pancreatic cancer, colorectal cancer, lung cancer and the like.

Other cancers that can be suitable treated with the compositions and methods of the present disclosure include, but are not limited to, acute myeloblastic leukemia (AML), acute lymphoblastic leukemia (ALL), chronic myelocytic leukemia (CML), adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, colon and rectum cancer, endometrial cancer, esophagus cancer, Ewing’s family of tumors (e.g. Ewing’s sarcoma), eye cancer, transitional cell carcinoma, vaginal cancer, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, gallbladder cancer, gastrointestinal carcinoid tumors, gastrointestinal stromal tumors, gestational trophoblastic disease, Non-Hodgkin’s lymphoma, Hodgkin’s lymphoma, childhood Non-Hodgkin’s lymphoma, Kaposi’s sarcoma, kidney cancer, laryngeal and hypopharyngeal cancer, liver cancer, lung cancer, lung carcinoid tumors, malignant mesothelioma, multiple myeloma, myelodysplastic syndrome, rhabdomyosarcoma, salivary gland cancer, sarcomas, melanoma skin cancer, non-melanoma skin cancers, stomach cancer, testicular cancer, thymus cancer, thyroid cancer, uterine cancer (e.g., uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom’s macroglobulinemia.

Particularly suitable cancers include, but are not limited to, breast cancer, ovarian cancer, lung cancer, pancreatic cancer, mesothelioma, leukemia, lymphoma, brain cancer, prostate cancer, multiple myeloma, melanoma, bladder cancer, bone sarcomas, soft tissue sarcomas, retinoblastoma, renal tumors, neuroblastoma, and carcinomas.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. For example, in some embodiments, the composition administered to the subject can reduce metastatic nodules in the subject. In some embodiments, the administered composition inhibits tumor growth in the subject.

In some embodiments, the proliferative disease is an autoimmune disease. In some embodiments, the autoimmune disease is selected from the group consisting of rheumatoid arthritis, insulin-dependent diabetes mellitus, hemolytic anemias, rheumatic fever, thyroiditis, Crohn’s disease, myasthenia gravis, glomerulonephritis, autoimmune hepatitis, multiple sclerosis, alopecia areata, psoriasis, vitiligo, dystrophic epidermolysis bullosa, systemic lupus erythematosus, moderate to severe plaque psoriasis, psoriatic arthritis, Crohn’s disease, ulcerative colitis, and graft vs. host disease.

In some embodiments, the administered composition inhibits proliferation of a target cancer cell, and/or inhibits tumor growth of the cancer in the subject. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cells disclosed herein, wherein the recombinant cells inhibit the proliferation of the target cell and/or inhibit tumor growth of a target cancer in the subject compared to the proliferation of the target cell and/or tumor growth of the target cancer in subjects who have not been administered with the recombinant cells.

Administration of the compositions described herein, e.g., ex vivo expanded T-cell populations and pharmaceutical compositions, can be used in the stimulation of an immune response. In some embodiments, ex vivo expanded T-cell populations and pharmaceutical compositions as described herein are administered to an individual after induction of remission of cancer with chemotherapy, or after autologous or allogeneic hematopoietic stem cell transplantation. In some embodiments, compositions described herein are administered to a subject in need of increasing the production of interferon gamma (IFNγ) and/or interleukin-2 (IL-2) in the treated subject relative to the production of these molecules in subjects who have not been administered one of the therapeutic compositions disclosed herein.

An effective amount of the compositions described herein, e.g., ex vivo expanded T-cell populations and/or pharmaceutical compositions, can be determined based on the intended goal, for example cancer regression. For example, where existing cancer is being treated, the amount of a composition disclosed herein to be administered may be greater than where administration of the composition is for prevention of cancer. One of ordinary skill in the art would be able to determine the amount of a composition to be administered and the frequency of administration in view of this disclosure. The quantity to be administered, both according to number of treatments and dose, also depends on the individual to be treated, the state of the individual, and the protection desired. Precise amounts of the composition also depend on the judgment of the practitioner and are peculiar to each subject. Frequency of administration could range from 1-2 days, to 2-6 hours, to 6-10 hours, to 1-2 weeks or longer depending on the judgment of the practitioner.

Determination of the amount of compositions to be administered will be made by one of skill in the art, and will in part be dependent on the extent and severity of cancer, and whether the recombinant cells are being administered for treatment of existing cancer or prevention of cancer. For example, longer intervals between administration and lower amounts of compositions may be employed where the goal is prevention. For instance, amounts of compositions administered per dose may be 50% of the dose administered in treatment of active disease, and administration may be at weekly intervals. One of ordinary skill in the art, in light of this disclosure, would be able to determine an effective amount of compositions and frequency of administration. This determination would, in part, be dependent on the particular clinical circumstances that are present (e.g., type of cancer, severity of cancer).

In some embodiments, it may be desirable to provide a continuous supply of a composition disclosed herein to the subject to be treated, e.g., a patient. In some embodiments, continuous perfusion of the region of interest (such as a tumor) may be suitable. The time period for perfusion would be selected by the clinician for the particular subject and situation, but times could range from about 1-2 hours, to 2-6 hours, to about 6-10 hours, to about 10-24 hours, to about 1-2 days, to about 1-2 weeks or longer. Generally, the dose of the composition via continuous perfusion will be equivalent to that given by single or multiple injections, adjusted for the period of time over which the doses are administered.

In some embodiments, administration is by intravenous infusion. An effective amount of the ex vivo expanded T cells disclosed herein can be determined based on the intended goal, for example tumor regression. For example, where existing cancer is being treated, the number of cells to be administered may be greater than where administration of the recombinant cells disclosed herein is for prevention of cancer. One of ordinary skill in the art would be able to determine the number of cells to be administered and the frequency of administration in view of this disclosure. The quantity to be administered, both according to number of treatments and dose, also depends on the individual to be treated, the state of the individual, and the protection desired. Precise amounts of the therapeutic composition also depend on the judgment of the practitioner and are peculiar to each individual. Frequency of administration could range from 1-2 days, to 2-6 hours, to 6-10 hours, to 1-2 weeks or longer depending on the judgment of the practitioner. Generally, the dose of the therapeutic composition via continuous perfusion will be equivalent to that given by single or multiple injections, adjusted for the period of time over which the doses are administered.

In some embodiments, the ex vivo expanded T cells disclosed herein are administered in a dosage of about 1,000 cells, 10,000 cells, 1×10³ cells, 1×10⁴ cells, 1×10⁵ cells, 1×10 ⁶ cells, 1×10⁷ cells or more, or in a range of about 1×10³ to 1×10⁴ cells, 1×10³ to 1×10⁵ cells, 1×10³ to 1×10⁶ cells, 1×10⁴ to 1×10⁵ cells, or 1×10⁵ to 1×10⁶ cells, 1×10⁶ to 1×10⁷ cells, 1×10⁷ to 1×10⁸ cells. In some embodiments, the ex vivo expanded T cells disclosed herein are administered in one or more dosages ranging from about 1×10⁵ cells to 1×10⁷ cells. In some embodiments, the ex vivo expanded T cells disclosed herein are administered in in one or more dosages of about 1×10⁶ cells. In some embodiments, the ex vivo expanded T cells disclosed herein are administered in one or more dosages ranging from about 1×10⁷ cells to 1×10¹⁰ cells. In some embodiments, the ex vivo expanded T cells disclosed herein are administered in a single administration. In some embodiments, cells are administered in multiple administrations, (e.g., once or more per week for one or more weeks). In some embodiments, doses are administered about every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more days. In some embodiments, there are 1, 2, 3, 4, 5, 6, 7, 8, 9, 10, or more total doses. In some embodiments, 4 doses are administered, with a 3 week span between doses.

One of ordinary skill in the art would be familiar with techniques for administering cells and cell-containing compositions to an individual. Furthermore, one of ordinary skill in the art would be familiar with techniques and pharmaceutical reagents necessary for preparation of these cells and cell-containing compositions prior to administration to an individual.

In certain embodiments of the present disclosure, the composition of the disclosure will be an aqueous composition that includes one or more of the ex vivo expanded T-cell populations and/or pharmaceutical compositions as described herein. Aqueous compositions of the present disclosure contain an effective amount of a composition disclosed herein in a pharmaceutically acceptable carrier or aqueous medium. Thus, the “pharmaceutical preparation” or “pharmaceutical composition” of the disclosure can include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the recombinant cells disclosed herein, its use in the manufacture of the pharmaceutical compositions is contemplated. Supplementary active ingredients can also be incorporated into the compositions. For human administration, preparations should meet sterility, pyrogenicity, general safety, and purity standards as required by the FDA Center for Biologics.

Upon formulation, the compositions of the disclosure will be administered in a manner compatible with the dosage formulation and in such amount as is therapeutically effective. The compositions can be administered in a variety of dosage forms, such as the type of injectable solutions described above. For parenteral administration, the compositions disclosed herein should be suitably buffered. As discussed in greater detail below, the compositions as described herein may be administered with other therapeutic agents that are part of the therapeutic regiment of the individual, such as other immunotherapy or chemotherapy. The ex vivo expanded T-cell populations and/or pharmaceutical compositions described herein can be used to inhibit tumor growth or metastasis of a cancer in the treated subject relative to the tumor growth or metastasis in subjects who have not been administered one of the therapeutic compositions disclosed herein. In some embodiments, the ex vivo expanded T-cell populations and/or pharmaceutical compositions described herein can be used to stimulate immune responses against the tumor via inducing the production of interferon gamma (IFNγ) and/or interleukin-2 (IL-2) and other pro-inflammatory cytokines. In some embodiments, the production of interferon gamma (IFNγ) and/or interleukin-2 (IL-2) can be stimulated to produce up to about 20 fold, such as any of about 2 fold, 3 fold, 4 fold, 5 fold, 6 fold, 7 fold, 8 fold, 9 fold, 10 fold, 11 fold, 12 fold, 13 fold, 14 fold, 15 fold 16 fold, 17 fold, 18 fold, 19 fold, or 20 fold or higher compared to the production of interferon gamma (IFNγ) and/or interleukin-2 (IL-2) in subjects who have not been administered one of the therapeutic compositions disclosed herein.

In some embodiments, the input T cells are obtained from an individual or subject. In some embodiments, the input T cells are autologous to the subject in need of treatment. In an autologous cell therapy, the ex vivo expanded T cells are administered to the individual that provided the input T cells.

In some embodiments, the input T cells are non-autologous to the subject in need of treatment. In some embodiments, the adoptive cell therapy is an allogeneic adoptive cell therapy. For example, in some embodiments, the input T cells are allogeneic to the subject in need of treatment. In an allogeneic adoptive cell therapy, the input T cells are not obtained from the individual receiving the adoptive cell therapy. Allogeneic cell therapy generally refers to a therapy whereby the individual (donor) who provides the input T cells is a different individual (of the same species) than the individual receiving the cell therapy. For example, an ex vivo expanded T population being administered to an individual is derived from one more unrelated donors, or from one or more non-identical siblings.

### Administration of Expanded T Cells to a Subject

In some embodiments, the methods of the disclosure involve administering an effective amount or number of the ex vivo expanded T cells as described herein to a subject in need of such treatment. This administering step can be accomplished using any method of implantation delivery in the art. For example, the expanded T cells can be infused directly in the subject’s bloodstream or otherwise administered to the subject.

In some embodiments, the methods disclosed herein include administering, which term is used interchangeably with the terms “introducing”, implanting”, and “transplanting”, expanded T cells into a subject, by a method or route that results in at least partial localization of the introduced cells at a desired site such that a desired effect(s) is/are produced. The expanded T cells can be administered by any appropriate route that results in delivery to a desired location in the subject where at least a portion of the administered cells or components of the cells remain viable. The period of viability of the cells after administration to a subject can be as short as a few hours, e.g., twenty-four hours, to a few days, to as long as several years, or even the lifetime of the subject, i.e., long-term engraftment.

When provided prophylactically, the expanded T cells described herein can be administered to a subject in advance of a symptom of a disease or condition to be treated. Accordingly, in some embodiments the prophylactic administration of an expanded T-cell population prevents the occurrence of symptoms of the disease or condition.

When provided therapeutically in some embodiments, expanded T cells are provided at (or after) the onset of a symptom or indication of a disease or condition, e.g., upon the onset of disease or condition.

For use in the various embodiments described herein, an effective amount of expanded T cells as disclosed herein, can be at least 10² cells, at least 5 × 10² cells, at least 10³ cells, at least 5 × 10³ cells, at least 10⁴ cells, at least 5 × 10⁴ cells, at least 10⁵ cells, at least 2 × 10⁵ cells, at least 3 × 10⁵ cells, at least 4 × 10⁵ cells, at least 5 × 10⁵ cells, at least 6 × 10⁵ cells, at least 7 × 10⁵ cells, at least 8 × 10⁵ cells, at least 9 × 10⁵ cells, at least 1 × 10⁶ cells, at least 2 × 10⁶ cells, at least 3 × 10⁶ cells, at least 4 × 10⁶ cells, at least 5 × 10⁶ cells, at least 6 × 10⁶ cells, at least 7 × 10⁶ cells, at least 8 × 10⁶ cells, at least 9 × 10⁶ cells, or multiples thereof.

In some embodiments, the delivery of an expanded T-cell composition (e.g., a composition including a plurality of expanded T cells as described herein) into a subject by a method or route results in at least partial localization of the cell composition at a desired site. A composition including expanded T cells can be administered by any appropriate route that results in effective treatment in the subject, e.g., administration results in delivery to a desired location in the subject where at least a portion of the composition delivered, e.g., at least 1 × 10⁴ cells, is delivered to the desired site for a period of time. Modes of administration include injection, infusion, instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a standard mode of administration.

In some embodiments, the expanded T cells are administered systemically, e.g., via infusion or injection. For example, a population of expanded T cells are administered other than directly into a target site, tissue, or organ, such that it enters, the subject’s circulatory system and, thus, is subject to metabolism and other similar biological processes.

The efficacy of a treatment including any of the expanded T cells and/or compositions provided herein for the treatment of a disease or condition can be determined by a skilled clinician. However, one skilled in the art will appreciate that a treatment is considered effective if any one or all of the signs or symptoms or markers of disease are improved or ameliorated. Efficacy can also be measured by failure of a subject to worsen as assessed by decreased hospitalization or need for medical interventions (e.g., progression of the disease is halted or at least slowed). Methods of measuring these indicators are known to those of skill in the art and/or described herein. Treatment includes any treatment of a disease in a subject or an animal (some non-limiting examples include a human, or a mammal) and can include: (1) inhibiting the disease, e.g., arresting, or slowing the progression of symptoms; or (2) relieving the disease, e.g., causing regression of symptoms; and (3) preventing or reducing the likelihood of the development of symptoms.

### Additional Therapies

As discussed above, any one of the compositions as disclosed herein, e.g., ex vivo expanded T-cell populations and pharmaceutical compositions, can be administered to a subject in need thereof as a single therapy (e.g., monotherapy). In addition or alternatively, in some embodiments of the disclosure, one or more of the T-cell populations and pharmaceutical compositions described herein can be administered to the subject in combination with one or more additional (e.g., supplementary) therapies, e.g., at least one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery. Other suitable therapies include therapeutic agents such as chemotherapeutics, anti-cancer agents, and anti-cancer therapies.

Administration “in combination with” one or more additional therapies includes simultaneous (concurrent) and consecutive administration in any order. In some embodiments, the one or more additional therapies is selected from the group consisting of chemotherapy, radiotherapy, immunotherapy, hormonal therapy, toxin therapy, and surgery. The term chemotherapy as used herein encompasses anti-cancer agents. Various classes of anti-cancer agents can be suitably used for the methods disclosed herein. Non-limiting examples of anti-cancer agents include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins such as irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple

### Podophyllum Peltatum

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell’s DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur. The main examples are vinca alkaloids and taxanes.

Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some embodiments, the anti-cancer agents can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof.

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some embodiments, the methods of prevention and/or treatment as described herein further include an immunotherapy. In some embodiments, the immunotherapy includes administration of one or more checkpoint inhibitors. Accordingly, some embodiments of the methods of treatment described herein include further administration of a compound that inhibits one or more immune checkpoint molecules. Non-limiting examples of immune checkpoint molecules include CTLA4, PD-1, PD-L1, A2AR, B7-H3, B7-H4, TIM3, and combinations of any thereof. In some embodiments, the compound that inhibits the one or more immune checkpoint molecules includes an antagonistic antibody. Examples of antagonistic antibodies suitable for the compositions and methods disclosed herein include, but are not limited to, ipilimumab, nivolumab, pembrolizumab, durvalumab, atezolizumab, tremelimumab, and avelumab.

In some aspects, the one or more anti-cancer therapy is radiation therapy. In some embodiments, the radiation therapy can include the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely, and depends on the half-life of the isotope and the strength and type of radiation emitted. When the radiation includes use of radioactive isotopes, the isotope may be conjugated to a targeting agent, such as a therapeutic antibody, which carries the radionucleotide to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). Removal of pre-cancers or normal tissues is also contemplated herein.

Accordingly, in some embodiments, the methods of the disclosure include administration of a composition disclosed herein to a subject individually as a single therapy (e.g., monotherapy). In some embodiments, a composition of the disclosure is administered to a subject as a first therapy in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are kits including the ex vivo expanded T cell populations and/or pharmaceutical compositions provided and described herein as well as written instructions for making and using the same. For example, provided herein, in some embodiments, are kits that include one or more ex vivo expanded T cell populations of the disclosure. In some embodiments, provided herein are kits that include one or more pharmaceutical compositions of the disclosure. In some embodiments, the kits of the disclosure further include one or more syringes (including pre-filled syringes) and/or catheters (including pre-filled syringes) used to administer one any of the provided recombinant nucleic acids, recombinant cells, or pharmaceutical compositions to a subject in need thereof.

In some embodiments, a kit can have one or more additional therapeutic agents that can be administered simultaneously or sequentially with the other kit components for a desired purpose, e.g., for modulating an activity of a cell, inhibiting a target cancer cell, or treating a health condition in a subject in need thereof.

For example, any of the above-described kits can further include one or more additional reagents, where such additional reagents can be selected from: dilution buffers; reconstitution solutions, wash buffers, control reagents, control expression vectors, negative control T-cell populations, positive control T-cell populations, reagents for ex vivo production of the T-cell populations.

In some embodiments, the components of a kit can be in separate containers. In some other embodiments, the components of a kit can be combined in a single container. For example, in some embodiments of the disclosure, the kit includes one or more of the provided ex vivo expanded T cell populations and/or pharmaceutical compositions as described herein in one container (e.g., in a sterile glass or plastic vial) and a further therapeutic agent in another container (e.g., in a sterile glass or plastic vial).

In some embodiments, a kit can further include instructions for using the components of the kit to practice the methods disclosed herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

Each of the aspects and embodiments described herein are capable of being used together, unless excluded either explicitly or clearly from the context of the embodiment or aspect.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

### Example 1

### High-Throughput Profiling of Ex Vivo Expanded T Cells Using CyTOF

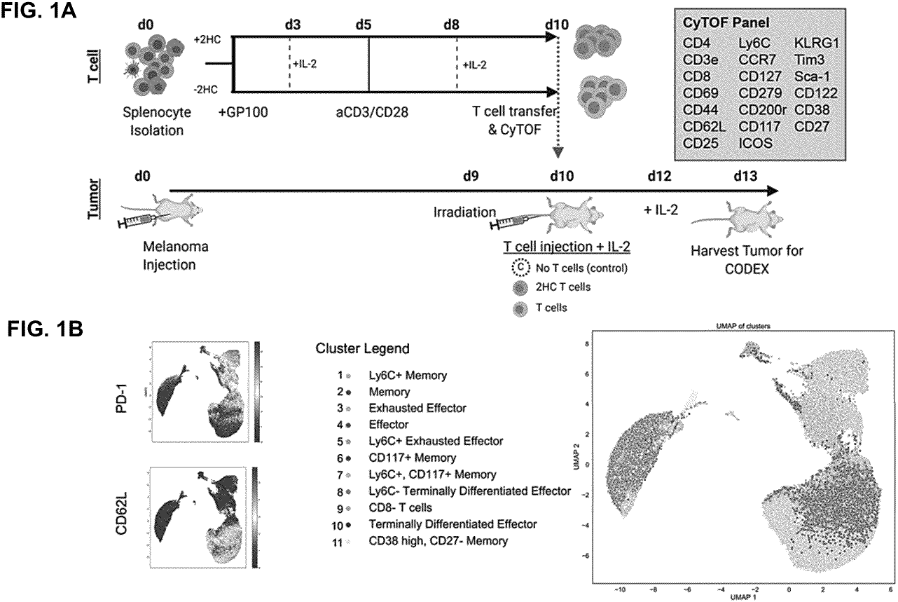

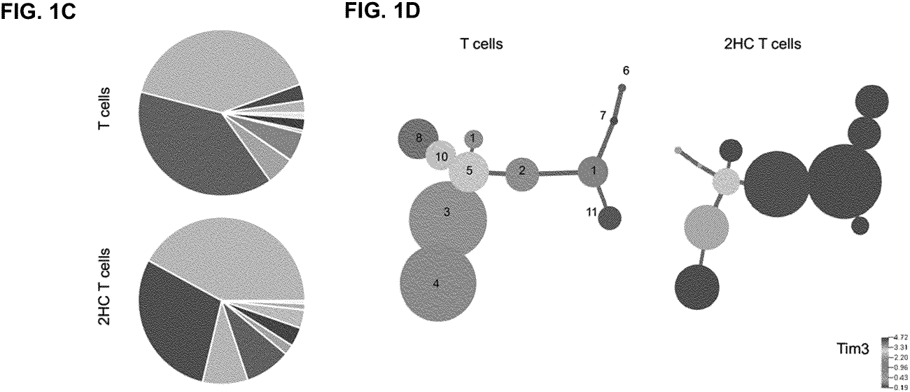

This Example describes experiments performed to apply the CyTOF technology to a comprehensively capture the phenotypic space of T cells. As described below, an in-depth understanding of the phenotype of differentially activated tumor-specific T cells can be achieved by using a CyTOF technique. This analysis revealed that by incubation of an inhibitor of acetyl-CoA production (e.g., metabolic inhibitor tripotassium 2-hydroxycitrate; “2HC”) drastically shifted T cells towards a more memory like phenotype whereas untreated cells displayed an effector and exhausted phenotype (see, e.g., FIG. 1). Also used in in these experiments was a palladium-based mass tag cell barcoding technique that enabled simultaneous analysis and pooling of 20 samples at a time, which allowed efficient multiplexing and reducing batch effects.

Generally, cells were suspended in supplemented media: complete RPMI 1640 media (with glutamine) with 1× non-essential amino acids, 1 mM sodium pyruvate, 0.4× vitamin solution, 92 µM 2-mercaptoethanol, 10 µM ciprofloxacin, 10% fetal bovine serum (FBS), and 50 IU/ml IL-2. At Day 0, splenocytes were used at a concentration of 2×10⁶/ml, 100 µL /well in 96 well plate u-bottom. The cultures were started with 100×10⁶ cells per condition, in 50 mL T cell media containing 60 IU/mL IL-2, 10 µg/mL GP100, and 5 mM 2HC (85 mg). At Day 3, the cell culture was fed with 50 µL media containing 120 IU/mL IL-2 and 5 mM 2HC. At Day 4, the cell culture was fed with 80 µL media containing 120 IU/mL IL-2 and 5 mM 2HC. At Day 5, the cultured cells were harvested, spun down, and resuspended cells in fresh media with 60 IU/mL and 1 ug/mL aCD28 (and 5 mM 2HC). Resuspended cells were moved to aCD3 plates (5 plates for each condition) in 100 µL per well. At Day 7, the cell culture was fed with 50 µL media containing 120 IU/mL IL-2 and 1 µg/mL aCD28 and 5 mM 2HC. At Day 8, the cells were moved to clean (i.e., containing no aCD3) plates with fresh media with 60 IU/mL IL-2 and 5 mM 2HC (10 plates per condition in 100 µL per well).

On Day 9, cells were counted and harvested into FACS tubes for CyTOF analysis using a panel of CYTOF biomarkers (see, e.g., FIG. 1 and Table 1). Approximately 4 million cells were used for staining per condition.

In these experiments, a panel of CYTOF biomarkers specific for murine T cell phenotype was developed (see, e.g., FIG. 1A and Table 1).

TABLE 1

Summary of CYTOF biomarkers and corresponding functionality

| Isotope | Antibody Target | Definition | T cell expression significance |
|---|---|---|---|
| 89 | CD178 | Fas Ligand - Induces apoptosis in other cell when binds to Fas | Help cell cytotoxicity |
| 113 | CD95 | Fas - induces apoptosis when binds to FasL | Indicative of T cell effector function |
| 115 | Ki67 | Proliferation | Indicative of T cell expansion and stimulation |
| 139 | Sca-1 | Broad stem cell marker | Indicative of high stemness, or ability to replicate/divide |
| 140 | CD122 | IL2/IL5 Receptor | Indicative of memory like phenotype |
| 141 | CD38 | cyclic ADP ribose hydrolase | Indicative of T cell activation and differentiation |
| 142 | CXCR5 | C-X-C chemokine receptor type 5 | Indicative of T cell functionality and ability to infiltrate |
| 143 | CD69 | C-Type lectin protein | Indicative of T cell activation |
| 144 | IL4 | Th2 cytokine | Helps promote more memory T cell formation |
| 145 | CD4 | CD4 helper cell marker | Expression differentiates from CD8+ T cells |
| 147 | CD27 | IL-7 receptor | Indicative of less differentiation, important cytokine for memory homeostasis |
| 148 | TCF-7 | Transcription factor 7 | Indicative of T cell stemness and functionality |
| 150 | CD25 | IL-2 Receptor alpha | Important cytokine for T cell activation and maintenance (induces proliferation) |
| 152 | CD3e | part of TCR | Indicative of T cell |
| 153 | IFNγ | Inflammatory cytokine | Indicates T cell functionality and cytotoxicity |
| 154 | Ly6C | GPI-anchored glycoprotein | Indicates further T cell differentiation |
| 155 | TNFα | Inflammatory cytokine | Indicates T cell functionality and cytotoxicity |
| 156 | CCR7 | C-C chemokine receptor type 7 | Indicative of more memory like phenotype |
| 157 | IL-2 | Inflammatory cytokine | Indicates T cell functionality and cytotoxicity, more expressed by memory cells and loss in transition to effector cells |
| 158 | CD127 | IL7 receptor | Indicative of more memory like cells - less differentiated |
| 159 | CD279 | PD1 | Indicative of T cell activation and high expression shows exhaustion |
| 160 | CD62L | L-selectin | Indicative of naive or memory like cells -less differentiated |
| 161 | CD107a | Lysosomal-associated membrane protein 1 | Indicative of T cell cytotoxicity |
| 162 | CD49b | Integrin alpha-2 | Indicative of short-lived T cells |
| 163 | granzyme b | Serine protease | Indicative of T cell cytotoxicity |
| 164 | CD335 | NKp46 | Marker for NK cells |
| 165 | perforin | pore-forming cytolytic protein | Indicative of T cell cytotoxicity |
| 166 | IL15 | Inflammatory cytokine | helps with T cell memory homeostasis |
| 167 | CD119 | IFNy Receptor | Receptor for IFNγ that regulates T cell proliferation and differentiation |
| 168 | CD8 | Cytotoxic T cell marker | Expression differentiates from CD4⁺ T cells |
| 169 | IL7 | Inflammatory cytokine | helps with T cell memory homeostasis |
| 170 | CD120b | TNFα Receptor 2 | Receptor for TNFα that regulates T cell proliferation and differentiation |
| 171 | CD44 | glycoprotein specific for HA | Indicative of potent effector functions |
| 173 | CD117 | Proto-oncogene c-KIT | Indicative of stem cell marker |
| 174 | KLRG1 | Killer cell lectin-like receptor subfamily G member 1 | Indicative of differentiation (less stemness) |
| 175 | Tim3 | Hepatitis A virus cellular receptor 2 | Indicative of T cell exhaustion |
| 176 | ICOS | Inducible T-cell costimulator | Indicative of T cell effector function and activation |

Also used in these experiments was a standard model of T cell therapies of PMEL T cells: transgenic mice where all TCRs are specific for the gp100 protein expressed by the melanoma cancer B16F10. This enabled not only standardization and cross-comparison between established literature but also the ability to mimic clinical protocols in solid tumors in mice. In these experiments, PMEL CD8⁺ T cells was stimulated for 10 days with 2-hydroxycitrate (5 mM, 2HC-treated T cells) and without 2-hydroxycitrate (T cells). It has been previously reported that 2-hydroxycitrate increases the generation of T memory stem cells by altering acetyl-Coenzyme A levels (see, e.g., FIG. 1A). On Day 10, stimulated T cells were isolated and transferred to B6 mice who had received B16-F10 melanoma cells 10 days earlier.

In addition, a portion of these cells was taken and stained with the T cell CyTOF panel of biomarkers. Analysis with unsupervised X-shift clustering revealed eleven distinct cell phenotypes within both conditions (see, e.g., FIG. 1B). These clusters separated out into largely four main populations of cells, as follows: (i) memory, (ii) effector, (iii) CD8⁻ cells, and (iv) exhausted effector. These eleven clusters can be visualized within a UMAP representation of all of the data combined (for both conditions: T cells and 2HC-treated T cells), and by overlaying exhaustion expression (PD-1) and memory expression (CD62L) revealed the accuracy of cluster identification.

Subsequently, the cluster proportion in both conditions was investigated. It was observed that the 2HC-treated T cells had a much higher proportion of memory like CD8⁺ T cells, whereas the untreated T cells had a much higher proportion of effector and exhausted phenotypes (see, e.g., FIG. 1C and Table 2).

TABLE 2

Exemplary cell composition profiles determined to be suitable for adoptive cell therapies

| | T cell populated 1 |
|---|---|
| T cell phenotype | Relative Proportion |
| Ly6C⁺ memory | 29.5% |
| Memory | 21.0% |
| Exhausted effector | 18.8% |
| Effector | 18.4% |
| Ly6C⁺ exhausted effector | 3.1% |
| CD117⁺ memory | 2.5% |
| Ly6C⁺, CD117⁺ memory | 2.4% |
| Ly6C⁻ terminally differentiated effector | 1.8% |
| CD8⁻ T cells | 1.0% |
| Terminally differentiated effector | 0.7% |
| CD38^{high}, CD27⁻ memory | 0.7% |

In these experiments, the cell composition profile includes relative proportions of the following cell subpopulations in the CD8⁺ T cells: (1) central memory, (2) stem cell memory, (3) effector memory, (4) naive, (5) effector, (6) terminally differentiated, and (7) exhausted (see, also Table 3).

TABLE 3

CD8⁺ T cell phenotypes included in an exemplary T cell population considered to be suitable for use in adoptive cell therapies

| CD8+ T cell Phenotypes | Core associated markers | | | | | | Other markers | | Proliferative Capacity | Cytotoxic Potential | Cytokine secretion ability |
|---|---|---|---|---|---|---|---|---|---|---|---|
| | CD62L | CD127 | CD44 | CD95 | CD27 | CCR7 | | | | | |
| Central Memory | high | high | high | low | high | high | | | High | Low | IL-2 high, others low |
| Stem Cell Memory | high | high | low | high | high | high | Sca-1 high | CD122 high | High | High | All high |
| Effector Memory | low | intermediate | high | intermediate | low | low | | | Intermediate | Intermediate | IFNg, INFa high |
| Naive | high | low | low | low | | | Ly6C low | | | | |
| Effector | low | low | high | high | low | low | | | Low | High | IFNg, INFa high |
| Terminally differentiated | low | low | high | | low | low | | | Low | Intermediate | low |
| Exhausted | low | low | high | | low | low | CD122 low | PD1 high | Low | Low | All low |

The relationships between the identified clusters with a trajectory inference algorithm of minimum spanning tree analysis was further analyzed (see, e.g., FIG. 1D). This analysis enabled identification of phenotypic nodes and comparison of conditions across this phenotypic space. In particular, this analysis identified four major branches, as follows: (1) terminally differentiated effector, (2) effector, (3) memory, and (4) CD117⁺ memory. Furthermore, analysis of cluster composition and overlaid expression of Tim3 demonstrate that phenotypically distinct populations have been identified for tumor treatment.

Without being bound to any particular theory, ex vivo expanded populations of T cells considered to be suitable for use in adoptive cell therapies are those having one or more of the following features: (i) high proliferative capacity characterized by memory markers such as e.g. CD122, CD27, CCR7, and CD127; (2) self-renewing capacity characterized by stem cell markers such as TCF7, Sca-1, CD117, and D62L; (3) high activation state characterized by markers such as CD69, CD44, ICOS, CD25, and CD38; (4) high functionality/cytotoxicity characterized by cytokines such as IFNy, TNFα, IL-2, CD95, and degranulation markers such as CD107a, perforin, granzyme b; and (5) low exhaustion profile characterized by low PD1, Tim3, KLRG1.

### Example 2

### Evaluation of Spatial Immune-Tumor and Immune-Immune Cell Interactions Using CODEX

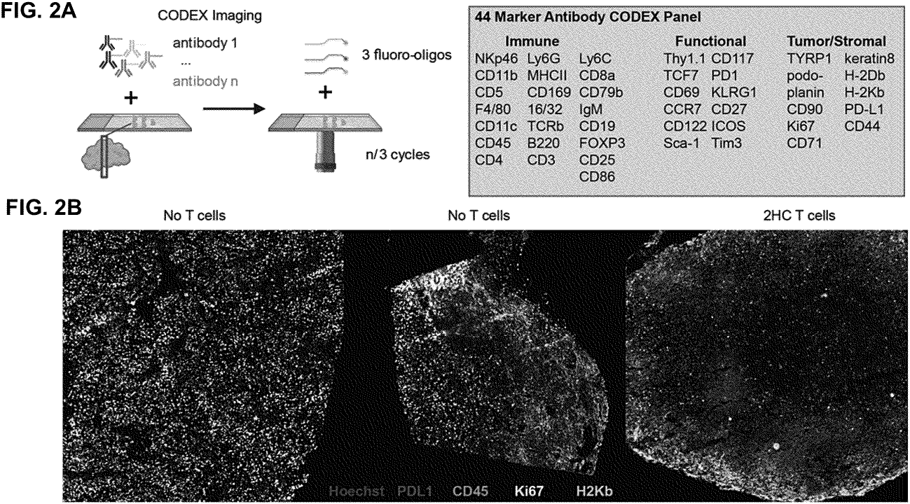

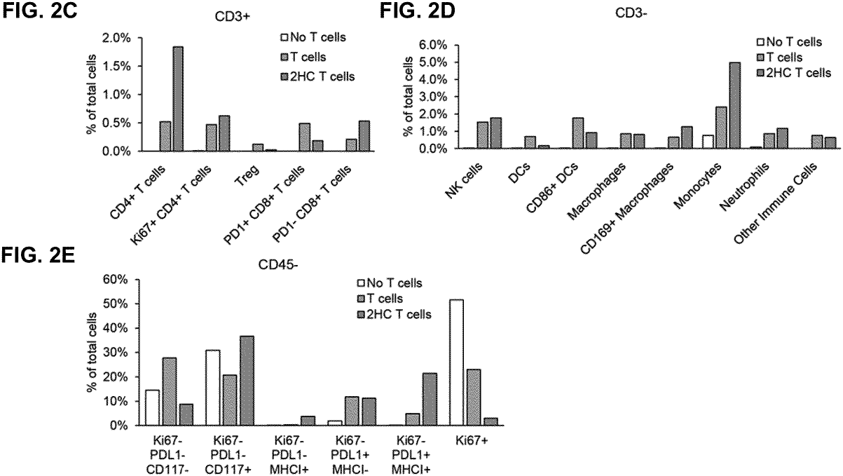

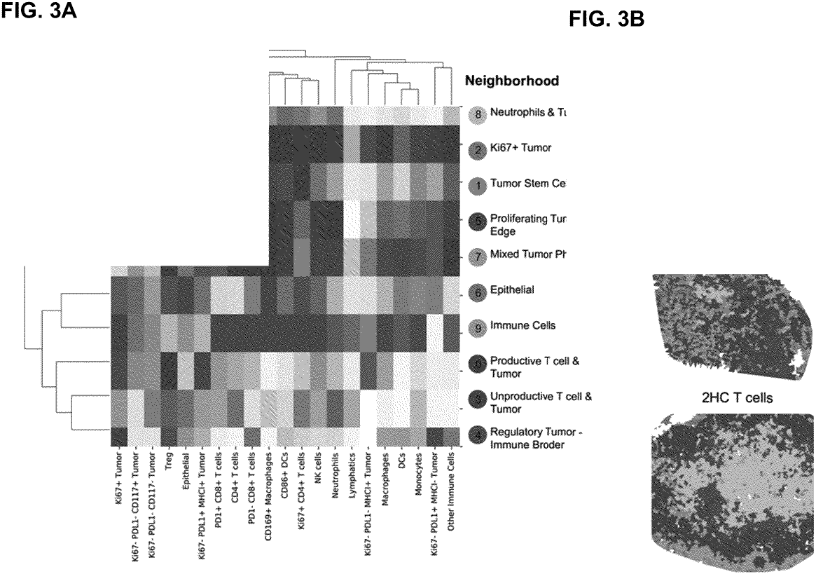

This Example described experiments performed to evaluate spatial immune-tumor and immune-immune cell interactions during treatment, by using the CODEX (CO-Detection by antibody indEXing) multi-parameter imaging technology. In these experiments, mice with established tumors were treated with the ex vivo expanded T cell population described in Example 1 (1 × 10 ⁶ cell/mouse), and harvested their tumors on day 3 and 5 for multiplexed CODEX imaging. Distinct recruitment of immune cells to the tumor from both treatment groups was observed, with higher total immune infiltrate from the 2HC T cell group and higher regulatory composition from the T cell group (see, e.g., FIGS. 2A-2E).

In these experiments, approximately 50 DNA-barcoded conjugated antibodies were used to stain tissues and then rendered iteratively with complementary fluorescent DNA probes (3 per imaging cycle) using an automated microfluidics system and imaged using a computer automated fluorescence microscope (see, e.g., FIG. 2A). This technology has enabled single-cell phenotyping at single-cell resolution. The combination of CyTOF and CODEX technologies in these experiments have provided several advantages. For example, the combination of CyTOF and CODEX technologies allowed to (i) study the dynamics of therapeutic immune cell phenotypes in vivo, (ii) evaluate cell-cell interactions key to influencing cell phenotype and function, and (iii) acquire a greater understanding of the composition of cellular neighborhoods necessary for mounting successful therapeutic immune cell therapies.

In particular, in these experiments, CODEX imaging technology was applied on the murine melanoma cancers (B16-F10) treated with cancer-specific CD8⁺ T cells (PMEL transgenic) which had been treated with and without 2HC (see, e.g., FIG. 1A). These were the same T cells analyzed on Day 10 with CyTOF described in Example 1 above. In particular, 1×10⁶ CD8⁺ T cells were adoptively transferred into each mouse with established tumors (>16 mm²). The tumors were then harvested either 3 or 5 days after treatment to make an array for CODEX imaging, including staining with a panel of 44 DNA-barcoded antibodies for immune, functional, stromal, and tumor-specific markers (see, e.g., FIG. 2A). The PMEL immune cells used in these experiments were transgenic for Thy1.1 marker (which is a highly expressed surface protein), while B6 wild-type immune cells express Thy1.2 marker, which allows for distinguishing transferred cells from native cells. This experimental design established an adoptive cellular therapy model that enabled direct insight to how changes in cell phenotype are linked to function.

It was observed that even by analyzing just 5 of our 44 markers, one could begin to see striking differences between the treated groups at Day 3: Hoechst (nuclear marker), PDL1 (tumor immunosuppresive marker), CD45 (immune marker), Ki67 (proliferation marker), H2Kb (MHC-I marker) (see, e.g., FIG. 2B). It was also observed that substantial upregulation of H2Kb and infiltration of CD45⁺ cells for both T cell treated melanomas over melanomas receiving no T cells. Additionally, it was also observed that melanoma treated with 2HC T cell has strikingly less Ki67⁺ tumor cells and a higher expression of PDL1 than either of the other melanomas. This result demonstrates a change in the tumor microenvironment based on the initial phenotype of the T cells that makes it less proliferative and more immuno-stimulatory.]

Additional experiments were performed to quantify percentages of each cell type by segmenting imaged cells based on nuclear staining. It was observed that there were almost no immune cells detected within the untreated tumor (see, e.g., FIGS. 2C and 2D). However, there was substantial immune infiltration into tumors from both tumor treatments (FIGS. 2C-2D). When comparing 2HC-treated T cells to T cell treated groups for CD3+ T cells, a significant increase in the percentage of CD4⁺ T cells and PD1- CD8⁺ T cells was observed, while there was a decrease in Treg and PD1⁺ CD8⁺ T cells—indicating more of a regulatory environment within the T cell treated group (see, e.g., FIG. 2C). Besides the recruitment of additional CD4⁺ T cells to the tumor, treated groups also recruited a substantial number of other immune cells including NK cells, DCs, macrophages, and neutrophils (see, e.g., FIG. 2D).

Additionally, significant differences in the profiles of different tumor phenotypes were also observed (see, e.g., FIG. 2E). Most drastic observation was the decrease in Ki67⁺ tumor cells by the T cell treated (~20%), and even more by the 2HC T cell treated (~3%) groups from the no treatment group (~50%). This observation indicates a drastic change in tumor growth program. Furthermore, also observed were increases in the Ki67- tumor cells that were expressing PDL1 and MHC-I, which are markers that indicate a proinflammatory environment. This observation indicates that the T cell therapy was having substantial effects on the cancer microenvironment, and that the memory-like phenotype of the 2HC-treated T cells was having substantial anti-proliferative and inflammatory effects on the cancer microenvironment.

### Example 3

### Computational Analysis of Immune-Tumor Neighborhoods From CODEX Data

This Example describes experiments performed to acquire further understanding of potential mechanisms for what might be happening to cause such striking differences in tumor program. As described in greater detail below, these experiments demonstrated a distinct decrease in proliferating (Ki67+) tumor cells with T cell treatment and almost complete reduction by 2HC T cell treated tumor cells. Subsequently, neighborhood analysis was performed and greatest conservation of structure and productive tumor-immune interactions was observed within the 2HC T cell treated tumors.

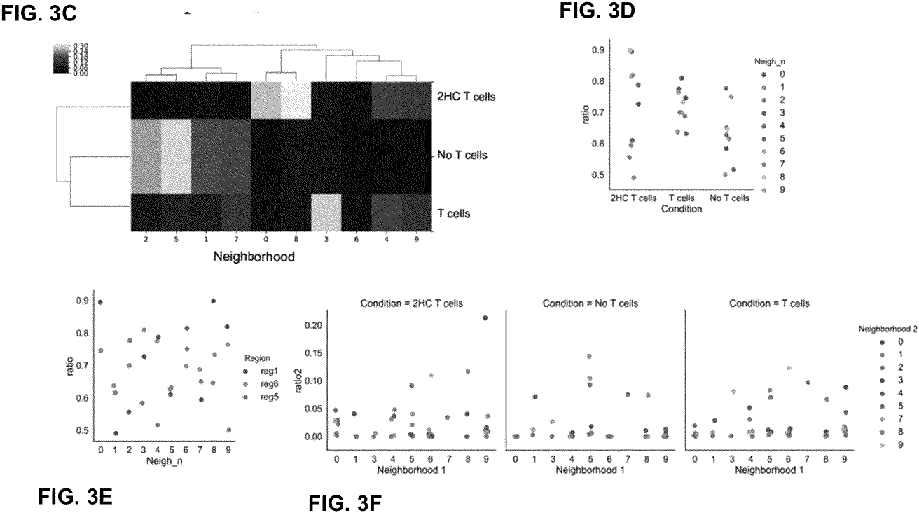

In these experiments, the spatial component of the data was used to probe what types of cellular neighborhoods existed in the data. The 10 nearest cell neighbors for each cell type were analyzed and then clustered these combinations to reveal 10 distinct neighborhoods (see, e.g., FIG. 3A). Neighborhoods 2, 1, 5, and 7 mostly comprised different tumor neighborhoods differentiated by whether there were proliferating, resting, or stem-cell like tumor cells. Neighborhood 6 and 9 were enriched for epithelial and immune cells. Neighborhoods 8, 0, 3, and 4 included a mixture of immune cells and tumor cells that were differentiated based on the immune cell and tumor composition: neutrophil enriched, productive T cell, unproductive T cell, and regulatory tumor-immune border.

It is clear from analyzing the neighborhoods plotted spatially from the original images as voronoi diagrams that there are both differences in neighborhood composition and organization (see, e.g., FIG. 3B). In particular, a much higher percentage of neighborhoods including tumor and proliferating tumor cells within the no-treated condition was observed, whereas there were a higher percentage of immune-tumor neighborhoods within the treated conditions. However, in the 2HC T cell condition, a greater enrichment for neighborhoods 0 and 8 (productive T cell, neutrophil-enriched) was observed, whereas in the T cell condition there was a greater enrichment for neighborhood 3 (unproductive T cell). Additionally, there was greater conservation between these productive neighborhoods forming a ring around the tumor. These enrichments were quantified as percentages within the heatmap of FIG. 3C.

What is equally striking is the level of organization between the different neighborhoods within each sample (see, e.g., FIG. 3B). Generally, the greatest organization was observed for 2HC treated tumors and the least organization was observed for untreated tumors. To quantify organization, the nearest neighbor for each cell type was examined to compute interactions between the cellular neighborhoods. Subsequently, the frequency of the interaction over the total number of cells within each neighborhood was evaluated and quantified by each treatment group. First, the conservation of neighborhoods, or the interaction of a neighborhood with its same neighborhood (e.g. neighborhood 5 with 5) was investigated (see, e.g., FIG. 3D). For the 2HC-treated T cell group, a high conservation between immune cell neighborhoods, 70%-90% (3, 4, 6, 9, 0, 8) was observed, while a low conservation of tumor neighborhoods, 40-60%, (7, 5, 2, 1) was observed. Higher conservation indicates higher levels of structure with the formation larger areas of self-contained neighborhoods. This is in direct contrast to the no treatment group, which followed the opposite relationship with immune cell neighborhoods having less organization, and overall has a lower overall inter-neighborhood conservation (see, e.g., FIG. 3D). Finally, there was intermediate level of organization of T cell treated group, with not much separation in the tumor vs. immune neighborhood group conservation. Furthermore, inter-neighborhood frequencies per treatment were separated out, a different conservation of tumor vs. immune organization between the treatments was observed (see, e.g., FIG. 3E).

Beyond neighborhood organization, the intra-neighborhood interactions were also examined (see, e.g., FIG. 3F). This analysis helped uncover whether there is further organization within the conservation of interactions at the borders of neighborhood. Within the 2HC T cell treated tumor, a conservation of a 0:9 (productive T cell:immune cells), 6:9 (epithelial:immune cells), and 8:1 (neutrophil-enriched:tumor stem cell) were observed. Particularly, the conservation of the 0:9 neighborhood interactions in the 2HC treated condition was higher than any other interaction across neighborhoods and treatments. For untreated tumors, a conservation of intra-tumoral borders was observed. In T cell treated tumors, a mix of the two phenotypes was again observed, while there was some conservation of immune intra-neighborhood interactions, but also tumor intra-neighborhood interactions, without as high enrichment for immune intra-neighborhood interactions as the 2HC-treated tumors.

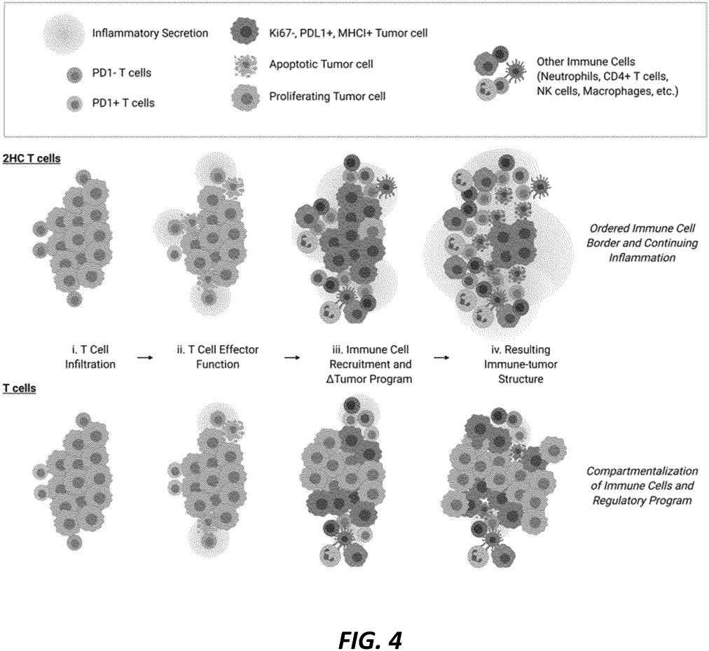

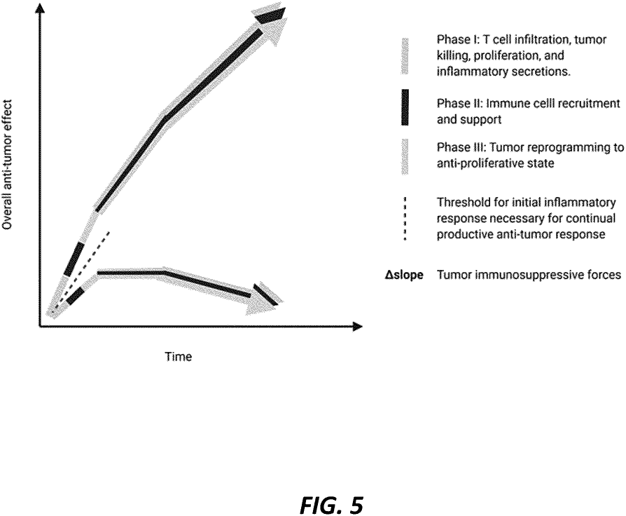

Based on these experimental results, several key conclusions can be drawn with regards to T cell treatment and successful immune-tumor reactions and a general cell model for is presented in FIG. 4. First, treated mice receive the same number of tumor-specific T cells (FIG. 4-i). However, 2HC-treated T cells have lower initial expression of effector and exhaustion markers like PD1 or Tim3. In both conditions, T cells will encounter tumor expressing cognate antigen and again become activated and proceed to kill (FIG. 4-ii). However, exhausted effector cells may be inhibited by checkpoint molecules already expressed on the tumor cell surface, so there is a reduced number of T cells that produce an effective killing response. Simultaneously, the activated T cells that are killing tumor are secreting proinflammatory molecules, which induces a) additional proliferation of T cells, b) recruitment of other immune cell types, and c) change in tumor program to stop proliferating (Ki67-) and increase expression of MHCI (antigen expression) and PDL1 (T cell inhibitory molecule) (FIG. 4-iii). Moreover, because there is a higher proportion of PD1- T cells in the 2HC T cell treatment group, the upregulation of PDL1 does not substantially affect the inflammatory response, whereas this upregulation will inhibit the majority of cells within the PD1+ T cells. Since there are a higher proportion of effective T cell-tumor interactions in the 2HC treated condition, this induces the recruitment of additional immune cells, increases inflammatory marker expression, and increases the reach of tumor reprogramming (FIG. 4-iv). However, because of the lower number of effective interactions in the T cell treated condition, this induces greater separation of the T cell/immune-tumor interactions, limits tumor reprogramming, and reduces further immune infiltration and proliferation. Consequently, there is no larger structure formed for T cell treated groups, while there is a productive advancing T cell/immune front in 2HC treated tumors.

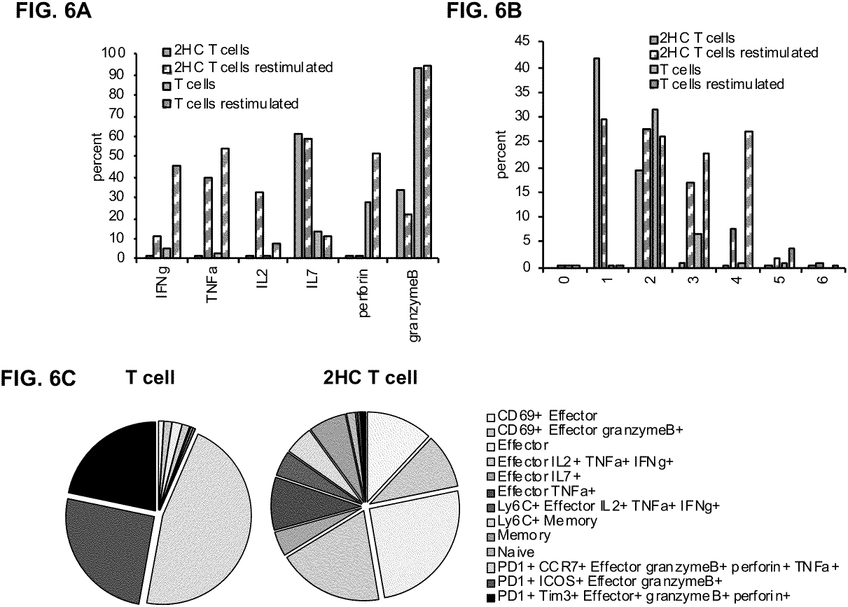

Two additional experiments were performed to investigate more specifically how the T cells influence the local microenvironment. In the first experiment, an intracellular cytokine CyTOF test was performed to look at the ability of stimulated and unstimulated cells to secrete effector molecules and is coupled with their phenotype. FIGS. 6A-6C graphically summarize the results of experiments illustrating intracellular cytokine staining for T cells treated with and without 2HC as measured by CyTOF.

Generally, the experiment was performed as follows. On Day 10 of culture, approximately 5,000 000 CD8+ T cells were isolated from each condition and separated into restimulation and no-stimulation groups in 100 µL T cell culture media (500,000 cells per well and 10 wells per condition). To inhibit protein transport, 1 µg anti-CD107 antibody, 1:350 BD GolgiStop Protein Transport Inhibitor (BD Biosciences), and 1:350 BD GolgiPlug Protein Transport Inhibitor (BD Biosciences) in PBS was added to the samples. Generally, for 5 × 10⁶ cells, 2.8 µL GolgiStop, 2.8 µL Golgi Plug, and 25 µL of anti-CD107 antibody were used. Both groups were then incubated at 37° C. for 6.5 hours. After incubation, cells were washed and then live/dead stain done (normal CyTOF protocol). After washing, cells with CSM were stained with surface antibodies (normal CyTOF stain protocol for about 30 minutes), then wash 1× in CSM, and 1× in PBS. Subsequently, cells were then fixed and permeabilized with 100 µL BD Cytofix/Cytoperm Fixation and Permeabilization Solution (BD Biosciences) overnight at 4° C.

To analyze intracellular cytokines, cells were washed with 1× BD PERM/Wash buffer with 2% bovine serum albumin (BSA) the following day, then cells were stained with in intracellular cytokine antibodies in PERM/Wash buffer with 2% BSA at 25° C. for 45 minutes. Cells were washed again in PBS and proceed to intercalation (normal protocol 30 min at RT) and washing (normal protocol).

As shown in FIG. 6A, it was observed that 2HC-treated cells secrete preferentially IL-7 and do so constitutively without need for stimulation and higher levels of IL-2 upon restimulation. Both molecules are needed for T cell maintenance, survival, and phenotype maintenance. Whereas T cells treated without 2HC produced more effector molecules (perforin, granzyme B, and IFNγ) indicating a more differentiated cell. FIG. 6B: Comparing the amounts of different cytokine and effector molecules secreted by the same cell shows that T cells without 2HC have a higher percentage of cells that secrete multiple effector molecules. FIG. 6C: However, zooming in with the phenotypic molecules used also within CyTOF, it was observed that T cells treated without 2HC that are secreting multiple effector molecules are also expressing cell exhaustion molecules like PD1 and Tim3 indicating late stage effector phenotype, whereas cytokine secreting cells in the 2HC condition are of memory phenotype. The results described herein indicate that as compared traditionally, T cells treated with 2HC in FIGS. 6A and 6B may not necessarily be classified as better than those treated with 2HC. However, by combining phenotype with secretion measurement, one can draw a conclusion that both measurements influence cell therapy outcome - T cell stage and molecular secretion. Furthermore, in the context of the spatial imaging results, one can further draw a conclusion that secretion of molecules such as IL-2 and IL-7 are necessary for the maintenance of the phenotype of the cells.

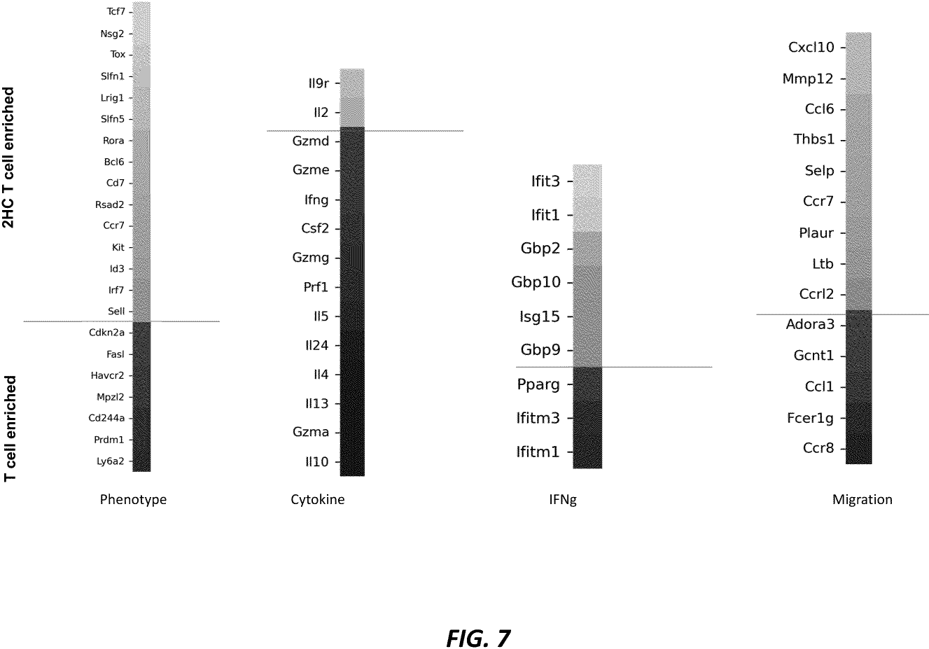

The second experiment was performed to follow up on the mechanism by which 2HC treated T cells create more productive tumor-immune microenvironments was to do RNA sequencing for both types of treated cells. RNA were isolated from samples using PureLink™ RNA Mini Kit (Thermo Fisher, Cat# 12183020). Generally, 1 µg of total RNA was used for subsequent steps. Isolation of poly(A) nRNA and library preparation were performed according to New England Biolabs procedures (NEB # E7760, E7490, and E7550). FIG. 7 graphically summarize the results of RNA-seq experiments performed on T cells treated differentially (e.g., with or without 2HC). Genes represented in green/yellow (above the horizontal line) are enriched for 2HC-treated T cells, while genes represented in blue (below the horizontal line) are enriched in T cells not treated with 2HC. Gene categories presented in this figure include (i) phenotype-associated genes, (ii) cytokine-associated genes, (iii) IFNγ-associated genes, and (iv) migration-associated genes. It was observed that phenotype and cytokine changes reflect differences observed within the CyTOF data already profiled. Cytokine differences also highlight the immuno-inhibitory cytokines also secreted by the cells not treated with 2HC. Migration differences reflect a differential ability of the 2HC-treated T cells to recruit specific cell types to the micro-environmental possibly pointing to a new metric to measure T cells appropriate for cell stimulation for cellular therapy.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

1 Yee C. Adoptive therapy using antigen-specific T-cell clones. Cancer J2010;16:367-73.

2 Yee C. The use of endogenous T cells for adoptive transfer. Immunol Rev 2014;257:250-63.

3 Restifo NP, Dudley ME, Rosenberg SA. Adoptive immunotherapy for cancer: harnessing the T cell response. Nat Rev Immunol 2012;12:269.

4 Baggio L, Laureano ÁM, da Rocha Silla LM, Lee DA. Natural killer cell adoptive immunotherapy: Coming of age. Clin Immunol 2017;177:3-11.

5 Vivier E, Ugolini S, Blaise D, Chabannon C, Brossay L. Targeting natural killer cells and natural killer T cells in cancer. Nat Rev Immunol 2012; 12:239-52.

6 Guillerey C, Huntington ND, Smyth MJ. Targeting natural killer cells in cancer immunotherapy. Nat Immunol 2016;17:1025.

7 Rezvani K, Rouce R, Liu E, Shpall E. Engineering natural killer cells for cancer immunotherapy. Mol Ther 2017;25:1769-81.

8 Johnson LA, June CH. Driving gene-engineered T cell immunotherapy of cancer. Cell Res 2017;27:38-58.

9 Siegel RL, Miller KD, Jemal A. Cancer statistics, 2019. CA Cancer J Clin 2019;69:7-34.

10 Ahmed N, Brawley VS, Hegde M, Robertson C, Ghazi A, Gerken C, et al. Human epidermal growth factor receptor 2 (HER2)-specific chimeric antigen receptor-modified T cells for the immunotherapy of HER2-positive sarcoma. J Clin Oncol 2015;33:1688.

11 Thistlethwaite FC, Gilham DE, Guest RD, Rothwell DG, Pillai M, Burt DJ, et al. The clinical efficacy of first-generation carcinoembryonic antigen (CEACAM5)-specific CAR T cells is limited by poor persistence and transient pre-conditioning-dependent respiratory toxicity. Cancer Immunol Immunother 2017;66:1425-36.

12 June CH, O′Connor RS, Kawalekar OU, Ghassemi S, Milone MC. CAR T cell immunotherapy for human cancer. Science (80-) 2018;359:1361-5.

13 Fesnak AD, June CH, Levine BL. Engineered T cells: The promise and challenges of cancer immunotherapy. NatRev Cancer 2016;16:566-81. https://doi.org/10.1038/nrc.2016.97.

14 Neelapu SS, Tummala S, Kebriaei P, Wierda W, Gutierrez C, Locke FL, et al. Chimeric antigen receptor T-cell therapy—assessment and management of toxicities. Nat Rev Clin Oncol 2018;15:47.

15 Ho WY, Yee C, Greenberg PD. Adoptive therapy with CD8+ T cells: it may get by with a little help from its friends. J Clin Invest 2002;110:1415-7.

16 Joyce JA, Fearon DT. T cell exclusion, immune privilege, and the tumor microenvironment. Science (80-) 2015;348:74-80.

17 Hegde M, Corder A, Chow KKH, Mukherjee M, Ashoori A, Kew Y, et al. Combinational targeting offsets antigen escape and enhances effector functions of adoptively transferred T cells in glioblastoma. Mol Ther 2013;21:2087-101.

18 Barrow C, Browning J, MacGregor D, Davis ID, Sturrock S, Jungbluth AA, et al. Tumor antigen expression in melanoma varies according to antigen and stage. Clin Cancer Res 2006;12:764-71.

19 Tumeh PC, Harview CL, Yearley JH, Shintaku IP, Taylor EJM, Robert L, et al. PD-1 blockade induces responses by inhibiting adaptive immune resistance. Nature 2014;515:568.

20 Verdegaal EME. Adoptive cell therapy: a highly successful individualized therapy for melanoma with great potential for other malignancies. Curr Opin Immunol 2016;39:90-5.

21 Bendall SC, Simonds EF, Qiu P, El-ad DA, Krutzik PO, Finck R, et al. Single-cell mass cytometry of differential immune and drug responses across a human hematopoietic continuum. Science (80-) 2011;332:687-96.

22 Good Z, Borges L, Gonzalez NV, Sahaf B, Samusik N, Tibshirani R, et al. Proliferation tracing with single-cell mass cytometry optimizes generation of stem cell memory-like T cells. Nat Biotechnol 2019:1.

23 Duckworth AD, Gherardini PF, Sykorova M, Yasin F, Nolan GP, Slupsky JR, et al. Multiplexed profiling of RNA and protein expression signatures in individual cells using flow or mass cytometry. Nat Protoc 2019;14:901-20.

24 Han G, Spitzer MH, Bendall SC, Fantl WJ, Nolan GP. Metal-isotope-tagged monoclonal antibodies for high-dimensional mass cytometry. Nat Protoc 2018;13:2121-48.

25 Goltsev Y, Samusik N, Kennedy-Darling J, Bhate S, Hale M, Vazquez G, et al. Deep profiling of mouse splenic architecture with CODEX multiplexed imaging. Cell 2018;174:968-81.

26 Aghaeepour N, Simonds EF, Knapp DJHF, Bruggner R V, Sachs K, Culos A, et al. GateFinder: projection-based gating strategy optimization for flow and mass cytometry. Bioinformatics 2018;34:4131-3.

27 Ko ME, Williams CM, Fread KI, Goggin SM, Rustagi RS, Fragiadakis GK, et al. FLOW-MAP: a graph-based, force-directed layout algorithm for trajectory mapping in single-cell time course datasets. Nat Protoc 2020:1-23.

28 Samusik N, Good Z, Spitzer MH, Davis KL, Nolan GP. Automated mapping of phenotype space with single-cell data. Nat Methods 2016;13:493.

29 Burns TJ, Nolan GP, Samusik N. Continuous visualization of differences between biological conditions in single-cell data. BioRxiv 2018:337485.

30 Henning AN, Roychoudhuri R, Restifo NP. Epigenetic control of CD8+ T cell differentiation. Nat Rev Immunol 2018;18:340.

31 Gattinoni L, Speiser DE, Lichterfeld M, Bonini C. T memory stem cells in health and disease. Nat Med2017;23:18.

32 Speiser DE, Utzschneider DT, Oberle SG, Münz C, Romero P, Zehn D. T cell differentiation in chronic infection and cancer: functional adaptation or exhaustion? Nat Rev Immunol 2014;14:768.

33 Speiser DE, Ho P-C, Verdeil G. Regulatory circuits of T cell function in cancer. Nat Rev Immunol 2016;16:599-611.

34 Kallies A, Zehn D, Utzschneider DT. Precursor exhausted T cells: key to successful immunotherapy? Nat Rev Immunol 2019:1-9.

35 Zunder ER, Finck R, Behbehani GK, El-ad DA, Krishnaswamy S, Gonzalez VD, et al. Palladium-based mass tag cell barcoding with a doublet-filtering scheme and single-cell deconvolution algorithm. Nat Protoc 2015;10:316.

36 Vodnala SK, Eil R, Kishton RJ, Sukumar M, Yamamoto TN, Ha N-H, et al. T cell stemness and dysfunction in tumors are triggered by a common mechanism. Science (80-) 2019;363:eaau0135.

37 Chen L, Flies DB. Molecular mechanisms of T cell co-stimulation and co-inhibition. Nat Rev Immunol 2013;13:227-42. https://doi.org/10.1038/nri3405.

38 Waldmann TA. The biology of interleukin-2 and interleukin-15: implications for cancer therapy and vaccine design. Nat Rev Immunol 2006;6:595-601. https://doi.org/10.1038/nri1901.

39 Fry TJ, Mackall CL. The many faces of IL-7: from lymphopoiesis to peripheral T cell maintenance. J Immunol (Baltimore, Md 1950) 2005;174:6571-6.

40 Gattinoni L, Zhong X-S, Palmer DC, Ji Y, Hinrichs CS, Yu Z, et al. Wnt signaling arrests effector T cell differentiation and generates CD8+ memory stem cells. Nat Med 2009;15:808.

41 Pollizzi KN, Patel CH, Sun I-H, Oh M-H, Waickman AT, Wen J, et al. mTORC1 and mTORC2 selectively regulate CD8+ T cell differentiation. J Clin Invest 2015;125:2090-108.

42 Chang C-H, Pearce EL. Emerging concepts of T cell metabolism as a target of immunotherapy. Nat Immunol 2016;17:364-8.

43 Sukumar M, Liu J, Ji Y, Subramanian M, Crompton JG, Yu Z, et al. Inhibiting glycolytic metabolism enhances CD8+ T cell memory and antitumor function. J Clin Invest 2013;123:4479-88.

44 Buchan SL, Manzo T, Flutter B, Rogel A, Edwards N, Zhang L, et al. OX40- and CD27-mediated costimulation synergizes with anti-PD-L1 blockade by forcing exhausted CD8+ T cells to exit quiescence. J Immunol 2015;194:125-33. https://doi.org/10.4049/jimmunol.1401644.

45 Hernandez-chacon JA, Li Y, Wu RC, Bernatchez C, Weber J, Hwu P, et al. Co-stimulation through the CD137/4-1BB pathway protects human melanoma tumor-infiltrating lymphocytes from activation-induced cell death and enhances anti-tumor effector function 2012;34:236-50. https://doi.org/10.1097/CJI.0b013e318209e7ec.Co-stimulation.

46 Chacon JA, Wu RC, Sukhumalchandra P, Molldrem JJ, Samaik A, Pilon-Thomas S, et al. Co-stimulation through 4-1BB/CD137 improves the expansion and function of CD8(+) melanoma tumor-infiltrating lymphocytes for adoptive T-cell therapy. PLoS One 2013;8:e60031. https://doi.org/10.1371/journal.pone.0060031.

47 Oh HS, Choi BK, Kim YH, Lee DG, Hwang S, Lee MJ, et al. 4-1BB Signaling Enhances Primary and Secondary Population Expansion of CD8+ T Cells by Maximizing Autocrine IL-2/IL-2 Receptor Signaling. PLoS One 2015;10:e0126765. https://doi.org/10.1371/journal.pone.0126765.

48 Rudolf D, Silberzahn T, Walter S, Maurer D, Engelhard J, Wernet D, et al. Potent costimulation of human CD8 T cells by anti-4-1BB and anti-CD28 on synthetic artificial antigen presenting cells. Cancer Immunol Immunother 2008;57:175-83. https://doi.org/10.1007/s00262-007-0360-x.

49 Zeng W, Su M, Anderson KS, Sasada T. Artificial antigen-presenting cells expressing CD80, CD70, and 4-1BB ligand efficiently expand functional T cells specific to tumor-associated antigens. Immunobiology 2014;219:583-92. https://doi.org/10.1016/j.imbio.2014.03.003.

50 Redmond WL, Linch SN, Kasiewicz MJ. Combined targeting of costimulatory (OX40) and coinhibitory (CTLA-4) pathways elicits potent effector T cells capable of driving robust antitumor immunity. Cancer Immunol Res 2014;2:142-53.

51 Hinrichs CS, Borman Z a, Gattinoni L, Yu Z, Burns WR, Klebanoff C a, et al. Human effector CD8 + T cells derived from naive rather than memory subsets possess superior traits for adoptive immunotherapy. October 2011;117:808-14. https://doi.org/10.1182/blood-2010-05-286286.

52 Klebanoff CA, Gattinoni L, Palmer DC, Muranski P, Ji Y, Hinrichs CS, et al. Determinants of successful CD8+ T-cell adoptive immunotherapy for large established tumors in mice. Clin Cancer Res 2011;17:5343-52.

53 Schietinger A, Philip M, Liu RB, Schreiber K, Schreiber H. Bystander killing of cancer requires the cooperation of CD4(+) and CD8(+) T cells during the effector phase. J Exp Med 2010;207:2469-77. https://doi.org/10.1084/jem.20092450.

54 Perret R, Ronchese F. Memory T cells in cancer immunotherapy: which CD8+ T-cell population provides the best protection against tumours? Tissue Antigens 2008;72:187-94.

55 Powell Jr DJ, Dudley ME, Robbins PF, Rosenberg SA. Transition of late-stage effector T cells to CD27+ CD28+ tumor-reactive effector memory T cells in humans after adoptive cell transfer therapy. Blood 2005;105:241-50.

56 Keren L, Bosse M, Marquez D, Angoshtari R, Jain S, Varma S, et al. A structured tumor-immune microenvironment in triple negative breast cancer revealed by multiplexed ion beam imaging. Cell 2018;174:1373-87.

57 Carstens JL, de Sampaio PC, Yang D, Barua S, Wang H, Rao A, et al. Spatial computation of intratumoral T cells correlates with survival of patients with pancreatic cancer. Nat Commun 2017;8:15095.

58 Kircher MF, Allport JR, Graves EE, Love V, Josephson L, Lichtman AH, et al. In vivo high resolution three-dimensional imaging of antigen-specific cytotoxic T-lymphocyte trafficking to tumors. Cancer Res 2003;63:6838-46.

59 Mandal A, Boopathy A V, Lam LKW, Moynihan KD, Welch ME, Bennett NR, et al. Cell and fluid sampling microneedle patches for monitoring skin-resident immunity. Sci Transl Med 2018;10: eaar2227.

60 Dieu-Nosjean M-C, Goc J, Giraldo NA, Sautès-Fridman C, Fridman WH. Tertiary lymphoid structures in cancer and beyond. Trends Immunol 2014;35:571-80.

61 Salerno EP, Shea SM, Olson WC, Petroni GR, Smolkin ME, McSkimming C, et al. Activation, dysfunction and retention of T cells in vaccine sites after injection of incomplete Freund’s adjuvant, with or without peptide. Cancer Immunol Immunother 2013;62:1149-59.

What is claimed is:

1. A method for preparing an ex vivo expanded T-cell population suitable for use in adoptive immunotherapy, the method comprising:
a) obtaining an input population of T cells from a subject;
b) expanding the obtained T cells in the presence of antigen stimulation and an inhibitor of acetyl-CoA production;
c) measuring the levels of a panel of biomarkers for T-cell specialization and/or exhaustion expressed in the expanded T-cell population to generate a cell composition profile; and
d) identifying the expanded T cell population as suitable for use in adoptive immunotherapy based at least in part upon the generated cell composition profile.

2. The method of claim 1, wherein the inhibitor of acetyl-CoA production is an inhibitor of ATP-citrate lyase (ACL), acyl-CoA synthetase short-chain family member 2 (ACSS2), carnitine acetyltransferase (CAT), and/or or pyruvate dehydrogenase complex (PDC).

3. The method of claim 2, wherein the ACL inhibitor is selected from the group consisting of hydroxycitric acid (HCA), bempedoic acid (ETC-1002), BMS-303141, SB 204990, and SB 201076, or a pharmaceutically acceptable salt thereof.

4. The method of any one of claims 2-3, wherein the ACL inhibitor comprises hydroxycitric acid (HCA) and/or a pharmaceutically acceptable salt thereof.

5. The method of claim 4, wherein the HCA salt is tripotassium 2-hydroxycitrate (2HC).

6. The method of any one of claims 1-5, wherein the inhibitor of acetyl-CoA production is present during expansion of the obtained T cells at a concentration ranging from 1 mM to 100 mM.

7. The method of any one of claims 1-5, wherein the panel of biomarkers for T-cell specialization and/or exhaustion comprises biomarkers representing one or more of the following features of the expanded T-cell population: proliferative potential, activated T cell, memory marker, further T cell differentiation, exhaustion, activation/memory/effector marker, stem cell memory marker, terminally differentiated marker, exhaustion marker, and T cell activation marker.

8. The method of any one of claims 1-7, wherein the panel of biomarkers for T-cell specialization and/or exhaustion comprises one or more of the following: CD3e, CD4, CD8, CD25, CD27, CD38, CD44, C62L, CD69, CD117, CD122, CD127, CD200r, CD279, CCR7, ICOS, KLRG1, Ly6C, Tim3, and Sca-1.

9. The method of any one of claims 1-8, wherein the cell composition profile comprises relative proportions of the following cell subpopulations: Ly6C⁺ memory cells, Ly6C⁺/CD117⁺ memory cells, memory cells, exhausted effector cells, effector cells, Ly6C⁺ exhausted effector cells, terminally differentiated effector cells, Ly6C⁻ terminally differentiated effector cells, CD8⁻ T cells, and CD3S^{high}/CD27⁻ memory cells.

10. The method of claim 9, the generation of the cell composition profile comprises using biomarkers that delimit substantially the same population as Ly6C⁺ memory cells, Ly6C⁺/CD117⁺ memory cells, memory cells, exhausted effector cells, effector cells. Ly6C⁺ exhausted effector cells, terminally differentiated effector cells, Ly6C⁻ terminally differentiated effector cells, CD8⁻ T cells, and CD38^{high}/CD27⁻ memory cells.

11. The method of any one of claims 1-10, further comprising measuring levels of one or more cytokines and/or effector molecules produced in the expanded T-cell population.

12. The method of any one of claims 1-11, wherein the step of obtaining an input population of T cells further comprises introducing into the T cells an immune receptor and/or nucleic acids encoding the immune receptor.

13. The method of claim 12, wherein the immune receptor is a T-cell receptor (TCR) or a chimeric antigen receptor (CAR).

14. The method of any one of claims 1-13, wherein the step of measuring the levels of a panel of biomarkers comprises using a nucleic-acid-based analytical assay selected from the group consisting of nucleic acid amplification-based assays, polymerase chain reaction (PCR), real-time PCR, nucleic acid sequencing, quantitative reverse transcription PCR (qRT-PCR), PCR-RFLP assay, HPLC, mass-spectrometric genotyping, nucleic acid hybridization assay, comparative genomic hybridization, restriction digestion, capillary electrophoresis, and combinations of any thereof.

15. The method of any one of claims 1-14, wherein the step of measuring the levels of a panel of biomarkers comprises using a protein-based analytical assay selected from the group consisting of immunohistochemistry (IHC), protein-microarray, western blotting, mass spectrometry, flow cytometry, enzyme-linked immunosorbent assay (ELISA), immunofluorescence staining, multiplex detection assay, and combinations of any thereof.

16. The method of any one of claims 1-15, further comprising harvesting the ex vivo expanded T cells.

17. The method of any one of claims 1-16, wherein the subject is a mammalian subject.

18. The method of any one of claims 1-17, wherein the subject has or is suspected of having a proliferative disorder, an autoimmune disorder, or an infection.

19. The method of claim 18, wherein the proliferative disorder is a cancer.

20. An ex vivo expanded T-cell population prepared by a method according to any one of claims 1-19.

21. The T-cell population of claim 20, wherein at least 60%, at least 70%, at least 80%, at least 90%, or at least 95% of the expanded T cells are memory T cells.

22. The T-cell population of claim 21, wherein the memory T cells comprise central memory cells, stem cell memory cells, and effector memory cells.

23. The T-cell population of any one of claims 21-22, wherein the memory cells comprise an increased expression of one of more biomarkers selected from the group consisting of CD62L, CD127, CD44, CD95, CD27, and CCR7, compared to control cells that are not cultured in the presence of the inhibitor of acetyl-CoA production.

24. The T-cell population of any one of claims 20-21, wherein the ratio of memory T cells to effector and/or exhausted T cells is about 2:1 to about 10:1.

25. The T-cell population of any one of claims 20-24, wherein the expanded T-cell population comprises a cell composition profile as set forth in Table 2 or Table 3.

26. The T-cell population of any one of claims 20-25, wherein the expanded T-cell population comprises one or more of the following characteristics: high proliferative capacity, self-renewing capacity, high activation state, high functionality/cytotoxicity, and low exhaustion profile.

27. A pharmaceutical composition comprising a T-cell population according to any one of claims 20-24, and a pharmaceutically acceptable excipient.

28. A method for preventing and/or treating a condition in a subject in need thereof, the method comprising administering to the subject a formulation comprising one or more of the following:
a) a T cell population according to any one of claims 20-24; and/or
b) a pharmaceutical composition according to any one of claims 27.

29. The method of claim 28, wherein the condition is a proliferative disorder, an autoimmune disorder, or an infection.

30. The method of any one of claims 28-29, wherein the T cells are autologous to the subject in need of treatment.

31. The method of any one of claims 28-29, wherein the T cells are allogeneic to the subject in need of treatment.

32. The method of any one of claims 28-31, wherein the subject is a mammalian subject.

33. The method of any one of claims 28-32, wherein the subject has or is suspected of having a proliferative disorder, an autoimmune disorder, or an infection.

34. The method of claim 33, wherein the proliferative disorder is a cancer.

35. The method of any one of claims 28-31, wherein the formulation is administered to the subject individually as a single therapy (monotherapy) or in combination with at least one additional therapies selected from the group consisting of chemotherapy, radiotherapy, immunotherapy, hormonal therapy, toxin therapy, targeted therapy, and surgery.

36. A kit for the prevention and/or treatment of a heath condition in a subject in need thereof, the kit comprising:
a) a T cell population according to any one of claims 20; and/or
b) a pharmaceutical composition according to any one of claims 27.
